(19) United States
(12) Patent Application Publication
(10) Pub. No.: US 2025/0230173 A1
(43) Pub. Date: Jul. 17, 2025

(54) SALT FORMS OF AN AXL INHIBITOR

(71) Applicant: Arcus Biosciences, Inc., Hayward, CA (US)

(72) Inventors: Jaroslaw Kalisiak, Newark, CA (US); Manmohan Reddy Leleti, Dublin, CA (US); Dillon Harding Miles, Berkeley, CA (US); Zhiguo Jake Song, Hayward, CA (US)

(73) Assignee: Arcus Biosciences, Inc., Hayward, CA (US)

(21) Appl. No.: 18/988,676

(22) Filed: Dec. 19, 2024

Related U.S. Application Data
(60) Provisional application No. 63/612,509, filed on Dec. 20, 2023.

(51) Int. Cl. C07D 519/00; A61K 45/06

(52) U.S. Cl. CPC C07D 519/00; A61K 45/06

(57) ABSTRACT

2-Naphthalene sulfonate salt forms of Compound (I), compositions containing those salt forms, and methods of preparation thereof are described herein. The use of the 2-naphthalene sulfonate salt forms of Compound (I), and associated compositions, for the treatment of diseases, disorders and conditions are also described.

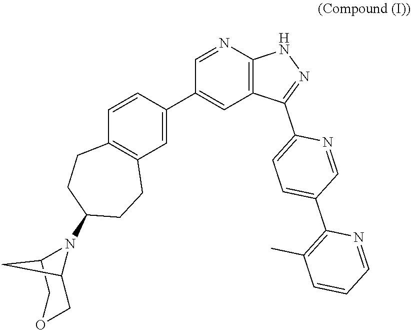 [SMILES: Cc1cccnc1-c1ccc(-c2n[nH]c3ncc(-c4ccc5c(c4)CC[C@@H](N4C6COCC4C6)CC5)cc23)nc1]

### CROSS-REFERENCE TO RELATED APPLICATIONS

This application claims the benefit of priority to U.S. Provisional Patent Application No. 63/612,509 filed on Dec. 20, 2023, the entire content of which is incorporated by reference herein.

### BACKGROUND OF THE DISCLOSURE

The following discussion is provided to aid the reader in understanding the disclosure and is not admitted to describe or constitute prior art thereto.

AXL is a receptor tyrosine kinase (RTK) that belongs to the TAM family. AXL regulates important processes such as cell growth, migration, aggregation, and apoptosis. AXL can be activated by a variety of mechanisms including ligand-dependent and ligand-independent mechanisms. Once activated AXL is involved in a variety of signaling pathways including the RAS-RAF-MEK-ERK pathway leading to cancer cell proliferation, and also the PI3K/AKT pathway responsible for several pro-survival proteins.

AXL has been shown to be overexpressed in a variety of malignancies. In cancer settings, AXL overexpression is associated with poor patient survival and resistance mechanisms (both targeted and non-targeted).

The compound 3-methyl-6′-{5-[(7S)-7-{3-oxa-6-azabicyclo[3.1.1]heptan-6-yl}-6,7,8,9-tetrahydro-5H-benzo[7]annulen-2-yl]-1H-pyrazolo[3,4-b]pyridin-3-yl}-2,3′-bipyridine, or alternatively 6-((S)-2-(3-(3-methyl-[2,3′-bipyridin]-6′-yl)-1H-pyrazolo[3,4-b]pyridin-5-yl)-6,7,8,9-tetrahydro-5H-benzo[7]annulen-7-yl)-3-oxa-6-azabicyclo[3.1.1]heptane designated herein as Compound (I), and having the structure:

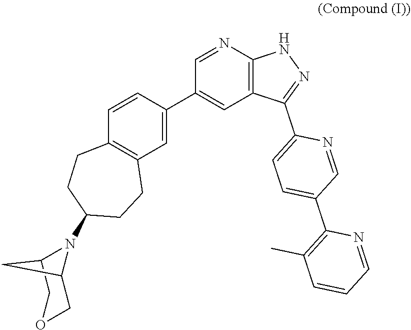 [SMILES: Cc1cccnc1-c1ccc(-c2n[nH]c3ncc(-c4ccc5c(c4)CC[C@@H](N4C6COCC4C6)CC5)cc23)nc1]

is a potent and highly selective inhibitor of AXL. In view of the role played by AXL in cancer, there is a need for solid forms of Compound (I) that possess desirable properties related to processing, manufacturing, storage, and/or usefulness as a drug, as well as compositions and methods associated therewith. The present disclosure addresses these needs and provides related advantages as well.

### BRIEF SUMMARY OF THE DISCLOSURE

In one aspect, this disclosure is directed to a 2-naphthalene sulfonate salt form of Compound (I):

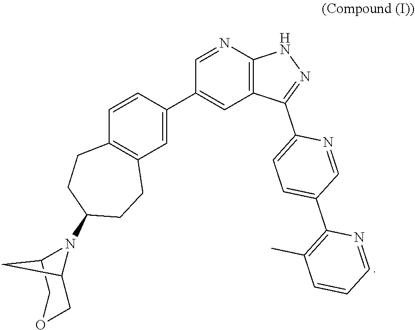 [SMILES: Cc1cccnc1-c1ccc(-c2n[nH]c3ncc(-c4ccc5c(c4)CC[C@@H](N4C6COCC4C6)CC5)cc23)nc1]

In another aspect, this disclosure is directed to pharmaceutical compositions comprising a 2-naphthalene sulfonate salt form of Compound (I) according to this disclosure.

In another aspect, this disclosure provides methods for treating a disease, disorder, or condition mediated at least in part by AXL in a subject, comprising administering to the subject a therapeutically effective amount of a 2-naphthalene sulfonate salt form of Compound (I) described herein, or a pharmaceutical composition comprising the same. Diseases, disorders, and conditions mediated by AXL include, e.g., cancer. Certain aspects of the present disclosure further comprise the administration of one or more additional therapeutic agents as set forth herein below.

Other objects, features, and advantages of the present disclosure will be apparent to one of skill in the art from the following detailed description and figures.

### BRIEF DESCRIPTION OF THE DRAWINGS

FIG. 1 depicts the XRPD pattern of Freebase Form I of Compound (I).

FIG. 2 depicts the XRPD pattern of Freebase Form II of Compound (I).

FIG. 3 depicts the XRPD pattern of Freebase Form III of Compound (I).

FIG. 4 depicts the XRPD pattern of 2-Napsylate Form I of Compound (I).

FIG. 5 depicts the XRPD pattern of 2-Napsylate Form II of Compound (I).

FIG. 6 depicts the XRPD pattern of 2-Napsylate Form III of Compound (I).

FIG. 7 depicts the XRPD pattern of 2-Napsylate Form III* of Compound (I).

FIG. 8 depicts the XRPD pattern of 2-Napsylate Form VI of Compound (I).

FIG. 9 depicts the XRPD pattern of Camsylate Form I of Compound (I).

FIG. 10 depicts the XRPD pattern of Fumarate Form I of Compound (I).

FIG. 11 depicts the XRPD pattern of Succinate Form I of Compound (I).

FIG. 12 depicts an overlay of the XRPD patterns of amorphous freebase of Compound (I) from the stability testing. Top is the XRPD pattern after storage for 7 days at 40° C./70% RH; second from top is the XRPD pattern after storage for 7 days at 25° C.; third from top is the XRPD pattern after storage form 7 days at 2° C.; and bottom is the XRPD pattern at the initial timepoint.

FIG. 13 depicts an overlay of the XRPD patterns of Freebase Form II of Compound (I) from the stability testing. Top is the XRPD pattern after storage for 7 days at 60° C.; second from top is the XRPD pattern after storage for 7 days at 40° C./70% RH; third from top is the XRPD pattern after storage for 7 days at 25° C.; and bottom is the XRPD pattern at the initial timepoint.

FIG. 14 depicts an overlay of the XRPD patterns of 2-Napsylate Form I of Compound (I) from the stability testing. Top is the XRPD pattern after storage for 7 days at 60° C.; second from top is the XRPD pattern after storage for 7 days at 40° C./70% RH; third from top is the XRPD pattern after storage for 7 days at 25° C.; and bottom is the XRPD pattern at the initial timepoint.

FIG. 15 depicts an overlay of the XRPD patterns of Fumarate Form I of Compound (I) from the stability testing. Top is the XRPD pattern after storage for 7 days at 40° C./70% RH; second from top is the XRPD pattern after storage for 7 days at 25° C.; third from top is the XRPD pattern after storage form 7 days at 2° C.; and bottom is the XRPD pattern at the initial timepoint.

FIG. 16 depicts an overlay of the XRPD patterns of Succinate Form I of Compound (I) from the stability testing. Top is the XRPD pattern after storage for 7 days at 40° C./70% RH; second from top is the XRPD pattern after storage for 7 days at 25° C.; third from top is the XRPD pattern after storage form 7 days at 2° C.; and bottom is the XRPD pattern at the initial timepoint.

FIG. 17 depicts an overlay of the XRPD patterns of Camsylate Form I of Compound (I) from the stability testing. Top is the XRPD pattern after storage for 7 days at 40° C./70% RH; second from top is the XRPD pattern after storage for 7 days at 25° C.; third from top is the XRPD pattern after storage form 7 days at 2° C.; and bottom is the XRPD pattern at the initial timepoint.

FIG. 18 depicts an overlay of the XRPD patterns for 2-Napsylate solid forms of Compound (I). Top is Form I; second from top is Form III/III*; third from top is Form IV; fourth from top is Form V; fifth from top is Form VI; sixth from top is Form VII; seventh from top is Form VIII; eighth from top is Form IX; and bottom is Form X.

FIG. 19A depicts an overlay of the XRPD patterns for 2-Napsylate Form I and 2-Napsylate Form VI of Compound (I); FIG. 19B depicts an overlay of the XRPD patterns for 2-Napsylate Form I and 2-Napsylate Form VI of Compound (I) with boxes around distinguishable regions.

FIG. 20 depicts an overlay of the XRPD patterns for 2-Napsylate Form I of Compound (I) under conditions of variable humidity at 25° C. Top is 2-Napsylate Form VI of Compound (I) reference; second from top is 10% RH; third from top is 20% RH; fourth from top is 30%; fifth from top is 40% RH; sixth from top is 50% RH; seventh from top is 60% RH; eighth from top is 70% RH; nineth from top is 80% RH; tenth from top is 90% RH; eleventh from top is 80% RH; twelfth from top is 70% RH; thirteenth from top is 60% RH; fourteenth from top is 50% RH; fifteenth from top is 40% RH; sixteenth from top is 30% RH; seventeenth from top is 20% RH; eighteenth from top is 10% RH; nineteenth from top (bottom plot) is 2-Napsylate Form I of Compound (I), reference.

FIG. 21 depicts an overlay of XRPD patterns of 2-Napsylate Form VI of Compound (I) before (red) and after (green) DVS testing.

FIG. 22 depicts an overlay of XRPD patterns of 2-Napsylate Form I of Compound (I) before (red) and after DVS testing (green).

FIG. 23 depicts an overlay of XRPD patterns of 2-Napsylate Form VI of Compound (I) before (red) and after (black) storage at 40° C. and 75% RH for one week.

FIG. 24 depicts an overlay of XRPD patterns of 2-Napsylate Form I of Compound (I) before (red) and after (black) storage at 40° C. and 75% RH.

FIG. 25 depicts the DSC thermogram of 2-Napsylate Form I of Compound (I).

FIG. 26 depicts the DSC thermogram of 2-Napsylate Form IV of Compound (I).

### DETAILED DESCRIPTION OF THE DISCLOSURE

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. It is also to be understood that recited ranges are inclusive of the upper and lower limits recited.

Unless defined otherwise, all technical and scientific terms used herein have the same meaning as commonly understood by one of ordinary skill in the art to which this disclosure belongs.

### Definitions

The term “about” as used herein has its original meaning of approximately and is to provide literal support for the exact number that it precedes, as well as a number that is near to or approximately the number that the term precedes. In general, the term “about” refers to the usual error range for the respective value readily known to the skilled person in this technical field, for example, the error range of a particular instrument or methodology. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “solvate” as used herein refers to forms of the compounds according to this disclosure (or the pharmaceutically acceptable salts thereof) that are associated with a solvent. Exemplary solvents that form solvates include, but are not limited to, water, methanol, ethanol, isopropanol, DMSO, 1,4-dioxane, ethyl acetate, acetic acid, and acetonitrile. Hydrates are included within the scope of the term “solvate” and refer to solvates wherein the compounds, or pharmaceutically acceptable salts thereof, are associated with water. The compound can be associated with the solvent in a stoichiometric or non-stoichiometric amount (e.g., hemihydrates, sesquihydrates, etc.).

The term “salt form” refers to an ionic compound containing cations and anions such that the net charge is neutral, i.e., the total positive charge balances the total negative charge. Salt forms can be formed by treating the neutral form (e.g., a freebase form) of a compound with an acid or a base. In certain aspects of this disclosure, the salt form is an acid addition salt form, i.e., a salt formed by treating a freebase compound with an acid. Salts can be dissolved in a solvent to make a solution, suspended in a solvent to result in a slurry, or be isolated in their solid form. Solid salt forms can be in the form of an amorphous form as defined herein, or a crystalline form as defined herein.

The term “solid form” as used herein refers to a form of a compound that has been isolated from solution using methods such as, for example, filtration, solvent evaporation, or combinations thereof. Solid forms can be in an amorphous form, crystalline form, or mixtures thereof.

The term “amorphous form” refers to a solid form of a compound having no definite crystal structure, i.e., lacking a regularly ordered, repeating pattern of constituent molecules.

The term “crystalline form” refers to a solid form of a compound wherein the constituent molecules are packed in a regularly ordered, repeating pattern. A crystalline form may refer to single-component or multi-component crystalline forms, for examples, including solvates, clathrates, and co-crystals.

The term “polymorph” refers to a crystalline form of a compound with a distinct spatial lattice arrangement as compared to other crystalline forms of the same compound. In general, different polymorphic or crystalline forms of a compound can be identified using methods such as X-ray powder diffraction (XRPD). The term “isomorphic” when referring to two or more crystalline forms indicates that the crystalline forms may contain different chemical moieties in the unit cell (e.g., may contain different solvent molecules), but are characterized by the same, or similar XRPD pattern.

“Substantially free” refers to an amount of 10% or less of another solid form or impurity, preferably 8%, 5%, 4%, 3%, 2%, 1%, 0.5%, or less of another form or impurity. In one embodiment, substantially free refers to a crystalline form of a compound of Compound (I) that contains less than 5% of other crystalline or amorphous forms of a compound of Compound (I). In one embodiment, substantially free refers to a crystalline form of a compound of Compound (I) that contains less than 1% of other crystalline or amorphous forms of a compound of Compound (I).

The terms “patient” or “subject” are used interchangeably to refer to a human or a non-human animal (e.g., a mammal). In one embodiment, the patient or subject is a human.

The terms “treat”, “treating”, treatment” and the like refer to a course of action that eliminates, reduces, suppresses, mitigates, ameliorates, or prevents the worsening of, either temporarily or permanently, a disease, disorder or condition to which the term applies, or at least one of the symptoms associated therewith. Treatment includes alleviation of symptoms, diminishment of extent of disease, inhibiting (e.g., arresting the development or further development of the disease, disorder or condition or clinical symptoms association therewith) an active disease, delaying or slowing of disease progression, improving the quality of life, and/or prolonging survival of a subject as compared to expected survival if not receiving treatment or as compared to a published standard of care therapy for a particular disease.

The term “in need of treatment” as used herein refers to a judgment made by a physician or similar professional that a subject requires or will benefit from treatment. This judgment is made based on a variety of factors that are in the realm of the physician's expertise, which may include a positive diagnosis of a disease, disorder or condition.

The terms “prevent”, “preventing”, “prevention”, “prophylaxis” and the like refer to a course of action initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state. Prevention also refers to a course of action initiated in a subject after the subject has been treated for a disease, disorder, condition or a symptom associated therewith in order to prevent relapse of that disease, disorder, condition or symptom.

### General

This disclosure is directed to solid forms of Compound (I) that have improved properties for their intended use. These properties may include improved physical and chemical stability, which may lend to improved manufacturing, processability, storage, and consistent properties when administered (such as absorption, bioavailability and other pharmacokinetic properties). As described herein, Compound (I), and pharmaceutically acceptable salts thereof, can exist in both amorphous and crystalline solid forms, the properties of which will be discussed in more detail below.

In one aspect this disclosure is directed to a 2-naphthalene sulfonate salt form of Compound (I)

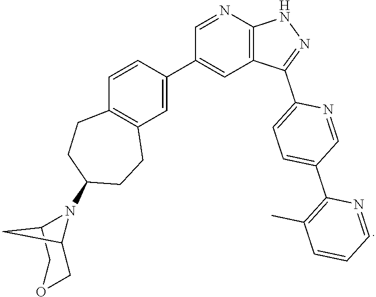 [SMILES: Cc1cccnc1-c1ccc(-c2n[nH]c3ncc(-c4ccc5c(c4)CC[C@@H](N4C6COCC4C6)CC5)cc23)nc1]

In one aspect the disclosure is directed to a 2-naphthalene sulfonate salt form of Compound (I) or a solvate thereof. In some embodiments, the 2-naphthalene sulfonate salt form is amorphous. In other embodiments, the 2-naphthalene sulfonate salt form is crystalline. The terms “2-naphthalene sulfonate salt”, and “2-Napsylate salt” are used interchangeably herein.

### Crystalline Forms

In some embodiments, the 2-naphthalene sulfonate salt form of Compound (I) is in a crystalline form. In some embodiments, the crystalline form is a solvate. In some embodiments, the crystalline form is a hydrate.

In some embodiments, the 2-Napsylate salt form of Compound (I) is 2-Napsylate Form I, 2-Napsylate Form II, 2-Napsylate Form III, 2-Napsylate Form III*, 2-Napsylate Form IV, 2-Napsylate Form V, 2-Napsylate Form VI, 2-Napsylate Form VII, 2-Napsylate Form VIII, 2-Napsylate Form IX, or 2-Napsylate Form X. In some embodiments, the crystalline form is 2-Napsylate Form I or 2-Napsylate Form VI. In some embodiments, the crystalline form is 2-Napsylate Form I. In some embodiments, the crystalline form is 2-Napsylate Form VI.

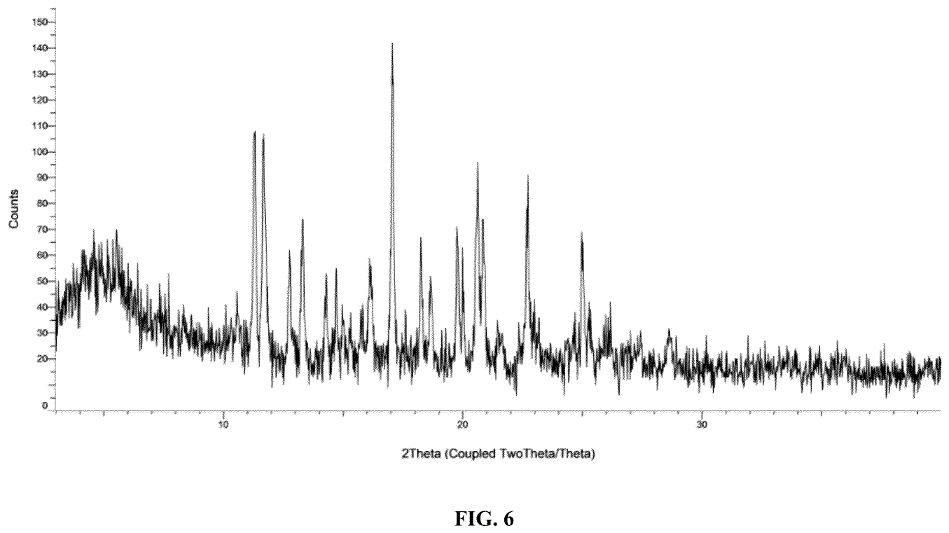

In some embodiments, the 2-Napsylate salt form of Compound (I) is 2-Napsylate Form II. In some embodiments, 2-Napsylate Form II is characterized by an XRPD pattern substantially in accordance with FIG. 5.

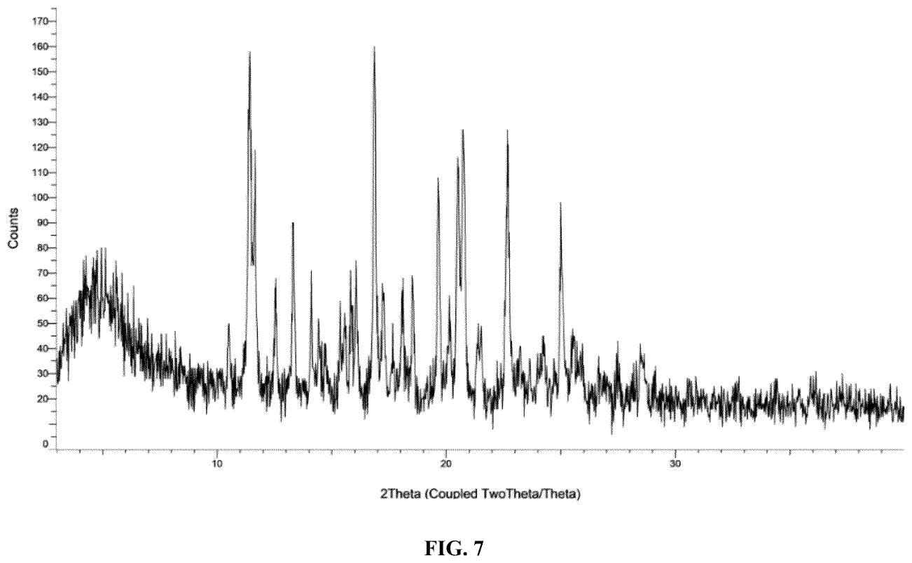

In some embodiments, the 2-Napsylate salt form of Compound (I) is 2-Napsylate Form III. In some embodiments, 2-Napsylate Form III is characterized by an XRPD pattern substantially in accordance with FIG. 6.

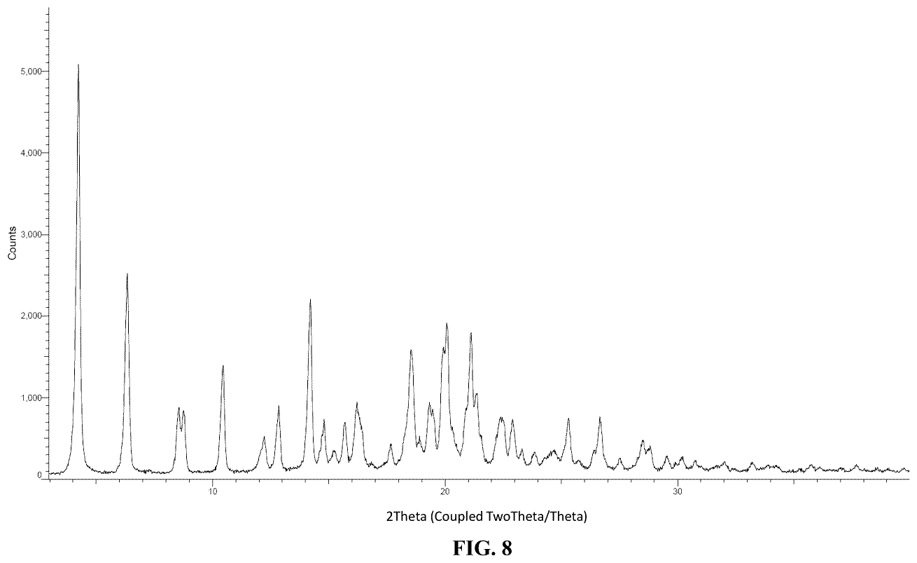

In some embodiments, the 2-Napsylate salt form of Compound (I) is 2-Napsylate Form III*. In some embodiments, 2-Napsylate Form III* is characterized by an XRPD pattern substantially in accordance with FIG. 7.

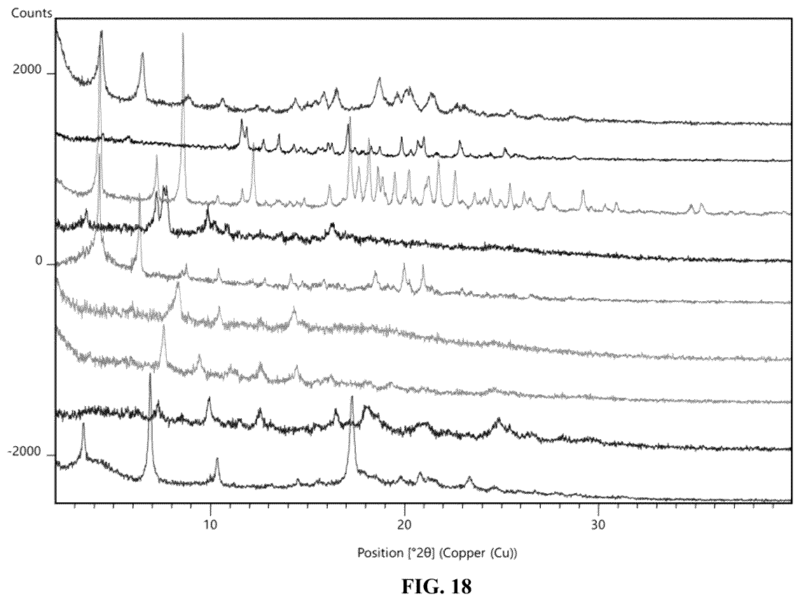

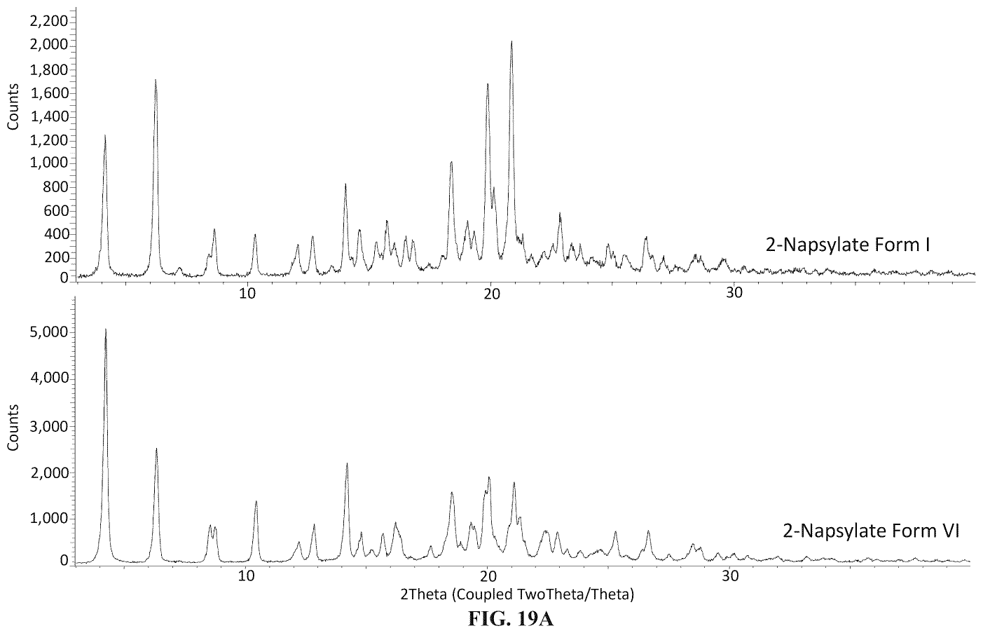

In some embodiments, the 2-Napsylate salt form of Compound (I) is 2-Napsylate Form IV. In some embodiments, 2-Napsylate Form IV is characterized by an XRPD pattern substantially in accordance with FIG. 18, third pattern from the top.

In some embodiments, the 2-Napsylate salt form of Compound (I) is 2-Napsylate Form V. In some embodiments, 2-Napsylate Form V is characterized by an XRPD pattern substantially in accordance with FIG. 18, fourth pattern from the top.

In some embodiments, the 2-Napsylate salt form of Compound (I) is 2-Napsylate Form VII. In some embodiments, 2-Napsylate Form VII is characterized by an XRPD pattern substantially in accordance with FIG. 18, sixth pattern from the top.

In some embodiments, the 2-Napsylate salt form of Compound (I) is 2-Napsylate Form VIII. In some embodiments, 2-Napsylate Form VIII is characterized by an XRPD pattern substantially in accordance with FIG. 18, seventh pattern from the top.

In some embodiments, the 2-Napsylate salt form of Compound (I) is 2-Napsylate Form IX. In some embodiments, 2-Napsylate Form IX is characterized by an XRPD pattern substantially in accordance with FIG. 18, eighth pattern from the top.

In some embodiments, the 2-Napsylate salt form of Compound (I) is 2-Napsylate Form X. In some embodiments, 2-Napsylate Form X is characterized by an XRPD pattern substantially in accordance with FIG. 18, bottom pattern. 2-Napsylate Form I

In some embodiments, the crystalline form is 2-Napsylate Form I. In some embodiments, 2-Napsylate Form I is characterized by XRPD signals at 4.2, 6.2, 18.4, 19.9, and 20.9 degrees 2θ (±0.2 degrees 2θ), as measured by XRPD using CuKα radiation. In some embodiments, 2-Napsylate Form I is further characterized by one or more, two or more, or three or more XRPD signals selected from the group consisting of 19.1, 19.4, 21.7, or 22.5 degrees 2θ (±0.2 degrees 2θ), as measured by XRPD using CuKα radiation. In some embodiments, 2-Napsylate Form I is further characterized by XRPD signals at 19.1, 19.4, 21.7, or 22.5 degrees 2θ (±0.2 degrees 2θ), as measured by XRPD using CuKα radiation. In some embodiments, 2-Napsylate Form I is characterized by XRPD signals at 4.2, 6.2, 18.4, 19.1, 19.4, 19.9, 20.9, 21.7, and 22.5 degrees 2θ (±0.2 degrees 2θ), as measured by XRPD using CuKα radiation.

In one embodiment, 2-Napsylate Form I is characterized by one or more XRPD signals at 4.2, 6.2, 18.4, 19.9, and 20.9 degrees 2θ (±0.2 degrees 2θ), as measured by XRPD using CuKα radiation. In still another embodiment, 2-Napsylate Form I is characterized by two, three, four or five XRPD signals at 4.2, 6.2, 18.4, 19.9, and 20.9 degrees 2θ (±0.2 degrees 2θ), as measured by XRPD using CuKα radiation. In some embodiments, 2-Napsylate Form I is further characterized by one or more, two or more, or three or more XRPD signals selected from the group consisting of 19.1, 19.4, 21.7, or 22.5 degrees 2θ (±0.2 degrees 2θ), as measured by XRPD using CuKα radiation.

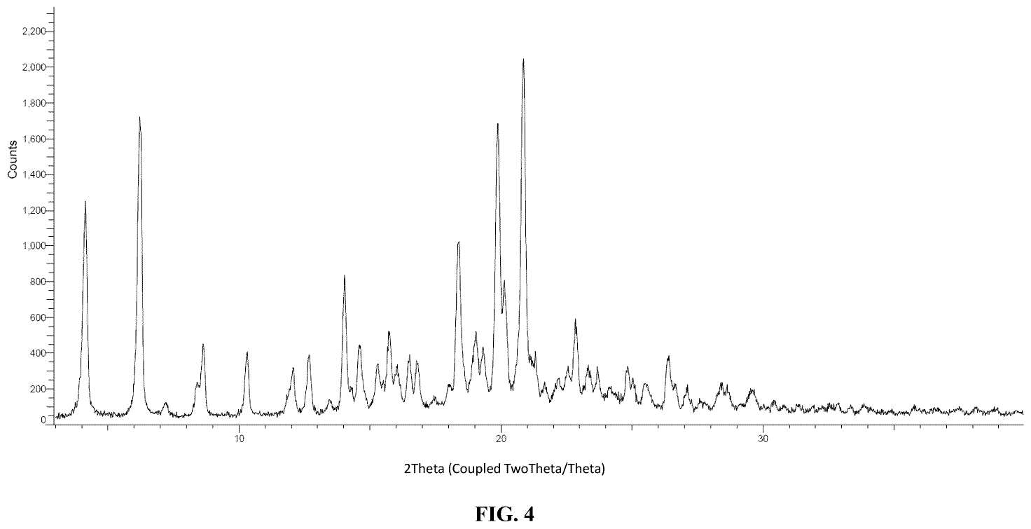

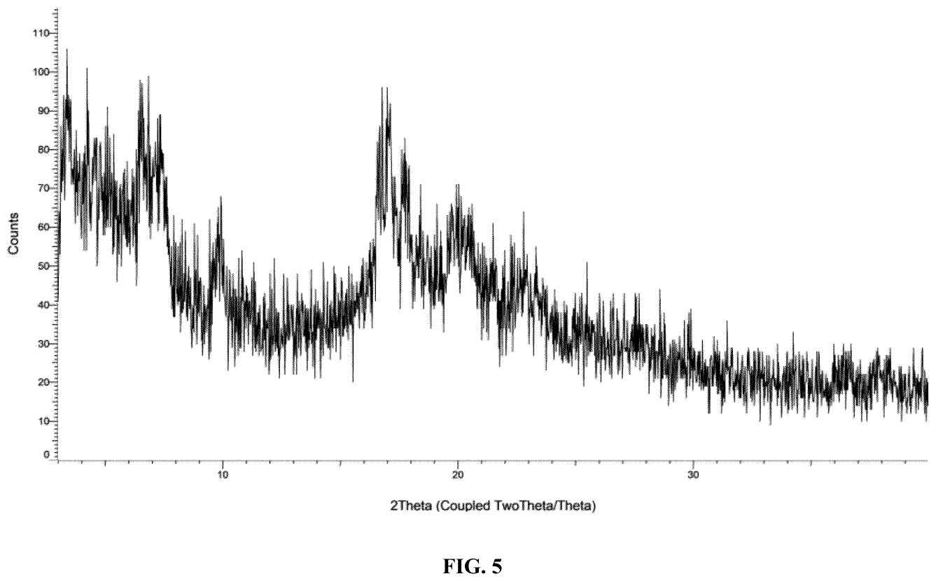

In some embodiments, 2-Napsylate Form I is characterized by an XRPD pattern substantially in accordance with FIG. 4.

In some embodiments, 2-Napsylate Form I is characterized by an XRPD pattern having one or more signals selected from those listed in Table 10, ±0.2 degrees 2θ, as measured using CuKα radiation.

In some embodiments, 2-Napsylate Form I is characterized by a differential scanning calorimetry (DSC) thermogram having an exotherm signal at about 247° C. In some embodiments, 2-Napsylate Form I is characterized by a differential scanning calorimetry (DSC) thermogram having an exotherm signal at 247° C. In some embodiments, 2-Napsylate Form I is characterized by a DSC thermogram having an exotherm signal between 230° C. and 260° C.

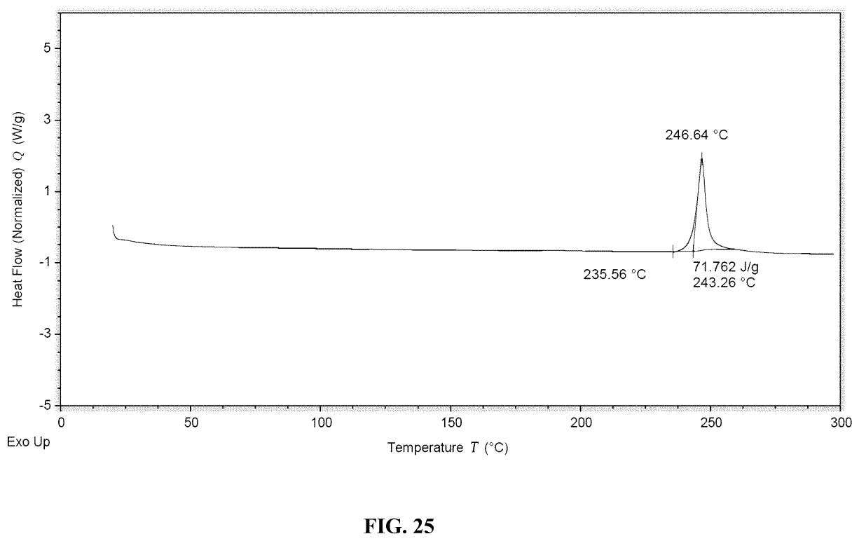

In some embodiments, 2-Napsylate Form I is characterized by a differential scanning calorimetry (DSC) thermogram substantially in accordance with FIG. 25.

### 2-Napsylate Form VI

In some embodiments, the crystalline form is 2-Napsylate Form VI. In some embodiments, 2-Napsylate Form VI is characterized by XRPD signals at 4.2, 6.3, 14.2, 20.1, and 21.1 degrees 2θ (±0.2 degrees 2θ), as measured by XRPD using CuKα radiation. In some embodiments, 2-Napsylate Form VI further characterized by one or more, two or more, or three or more XRPD signals selected from the group consisting of 12.2, 21.4, 25.3, and 26.7 degrees 2θ (±0.2 degrees 2θ), as measured by XRPD using CuKα radiation. In some embodiments, 2-Napsylate Form VI is further characterized by XRPD signals at 12.2, 21.4, 25.3, and 26.7 degrees 2θ (±0.2 degrees 2θ), as measured by XRPD using CuKα radiation. In some embodiments, 2-Napsylate Form VI is characterized by XRPD signals at 4.2, 6.3, 12.2, 14.2, 20.1, 21.1, 21.4, 25.3, and 26.7 degrees 2θ (±0.2 degrees 2θ), as measured by XRPD using CuKα radiation.

In one embodiment, the 2-Napsylate Form VI is characterized by one or more XRPD signals at 4.2, 6.3, 14.2, 20.1, and 21.1 degrees 2θ (±0.2 degrees 2θ), as measured by XRPD using CuKα radiation. In still another embodiment, 2-Napsylate Form VI is characterized by two, three, four or five XRPD signals at 4.2, 6.3, 14.2, 20.1, and 21.1 degrees 2θ (±0.2 degrees 2θ), as measured by XRPD using CuKα radiation. In some embodiments, 2-Napsylate Form VI further characterized by one or more, two or more, or three or more XRPD signals selected from the group consisting of 12.2, 21.4, 25.3, and 26.7 degrees 2θ (±0.2 degrees 2θ), as measured by XRPD using CuKα radiation.

In some embodiments, the 2-Napyslate Form VI is characterized by an XRPD pattern substantially in accordance with FIG. 8.

In some embodiments, 2-Napsylate Form VI is characterized by an XRPD pattern having one or more signals selected from those listed in Table 11, ±0.2 degrees 2θ, as measured using CuKα radiation.

In some embodiments, 2-Napsylate Form VI is characterized by a differential scanning calorimetry (DSC) thermogram having an exotherm signal at about 243° C. In some embodiments, 2-Napsylate Form VI is characterized by a differential scanning calorimetry (DSC) thermogram having an exotherm signal at 243° C. In some embodiments, 2-Napsylate Form VI is characterized by a DSC thermogram having an exotherm signal between 225° C. and 275° C.

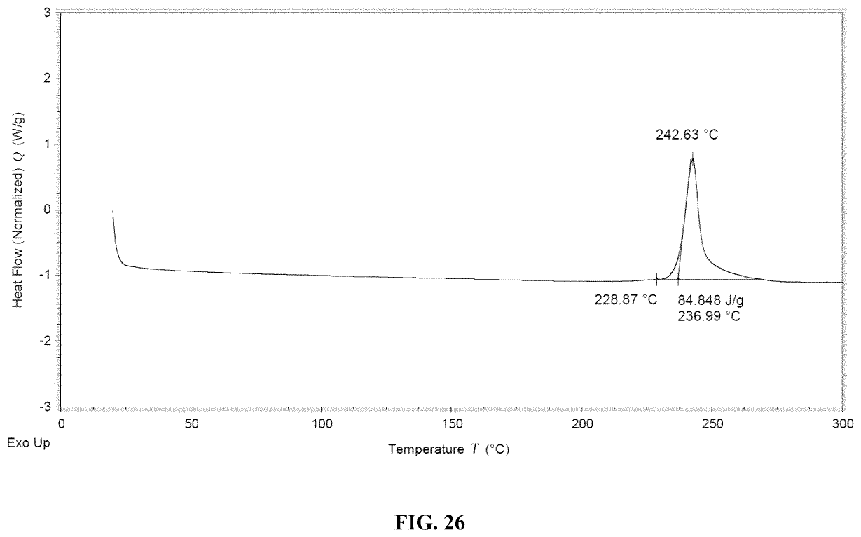

In some embodiments, 2-Napsylate Form VI is characterized by a differential scanning calorimetry (DSC) thermogram substantially in accordance with FIG. 26.

### Stability

In some embodiments, the crystalline 2-naphthalene sulfonate salt form of Compound (I) according to this disclosure has improved stability relative to other solid forms of Compound (I), i.e., amorphous forms or crystalline forms that are not 2-naphthalene sulfonate salt forms of Compound (I). In some embodiments, the 2-naphthalene sulfonate salt form of Compound (I) has improved chemical stability and/or physical stability. In some embodiments, the 2-naphthalene sulfonate salt form of Compound (I) has improved chemical stability. In some embodiments, the 2-naphthalene sulfonate salt form of Compound (I) has improved physical stability.

High performance liquid chromatography (HPLC) is a method that can be used, for example, to assess the chemical stability of a solid form after storage under a variety of conditions. Without wishing to be bound by theory, a decrease in purity as determined by HPLC indicates that chemical decomposition has occurred. In some embodiments, the purity of the crystalline 2-naphthalene sulfonate salt form of Compound (I) (e.g., 2-Napsylate Form I) remains substantially unchanged as measured by HPLC when stored for a prolonged period of time. Substantially unchanged, in this context means less than 1%, less than 0.7%, less than 0.5%, less than 0.4%, less than 0.3%, less than 0.2%, or less than 0.1%. In some embodiments, the purity of the crystalline 2-naphthalene sulfonate salt form of Compound (I) (e.g., 2-Napsylate Form I) remains unchanged as measured by HPLC when stored at a temperature of about 25° C. for a period of at least 7 days. In some embodiments, the purity of the crystalline 2-naphthalene sulfonate salt form of Compound (I) (e.g., 2-Napsylate Form I) decreases by less than about 1% as measured by HPLC when stored at a) a temperature of about 25° C. for at least 7 days; b) a temperature of about 60° C. for at least 7 days; and/or c) a temperature of about 40° C. and a relative humidity of about 75% for a period of at least 7 days. In some embodiments, the purity of the crystalline 2-naphthalene sulfonate salt form of Compound (I) (e.g., 2-Napsylate Form I) decreases by less than about 0.7% as measured by HPLC when stored at a) a temperature of about 25° C. for at least 7 days; b) a temperature of about 60° C. for at least 7 days; and/or c) a temperature of about 40° C. and a relative humidity of about 75% for a period of at least 7 days. In some embodiments, the purity of the 2-naphthalene sulfonate salt form of Compound (I) (e.g., 2-Napsylate Form I) decreases by less than about 0.5% as measured by HPLC when stored at a) a temperature of about 25° C. for a period of at least 7 days; and/or b) a temperature of about 40° C. and a relative humidity of about 75% for a period of at least 7 days. In some embodiments, the purity of the 2-naphthalene sulfonate salt form of Compound (I) (e.g., 2-Napsylate Form I) decreases by less than about 1% as measured by HPLC when stored at a) a temperature of about 25° C. and a relative humidity of 60% for a period of at least 1 month; and/or b) a temperature between about 2° C. and about 8° C. for a period of at least 1 month. In some embodiments, the purity of the 2-naphthalene sulfonate salt form of Compound (I) (e.g., 2-Napsylate Form I) decreases by about 5% or less as measured by HPLC, when stored at a) a temperature of about 25° C. and a relative humidity of about 60% for a period of at least 1 month (e.g., at least 3 months, at least 6 months, at least 9 months, at least 12 months, at least 18 months, at least 24 months, or at least 36 months); and/or b) a temperature between about 2° C. and about 8° C. for a period of at least 1 month (e.g., at least 3 months, at least 6 months, at least 9 months, at least 12 months, at least 18 months, at least 24 months, or at least 36 months). In some embodiments, the purity of the 2-naphthalene sulfonate salt form of Compound (I) (e.g., 2-Napsylate Form I) decreases by about 3% or less as measured by HPLC, when stored at a) a temperature of about 25° C. and a relative humidity of about 60% for a period of at least 1 month (e.g., at least 3 months, at least 6 months, at least 9 months, at least 12 months, at least 18 months, at least 24 months, or at least 36 months); and/or b) a temperature between about 2° C. and about 8° C. for a period of at least 1 month (e.g., at least 3 months, at least 6 months, at least 9 months, at least 12 months, at least 18 months, at least 24 months, or at least 36 months). In some embodiments, the purity of the 2-naphthalene sulfonate salt form of Compound (I) (e.g., 2-Napsylate Form I) decreases by about 1% or less as measured by HPLC, when stored at a) a temperature of about 25° C. and a relative humidity of about 60% for a period of at least 1 month (e.g., at least 3 months, at least 6 months, at least 9 months, at least 12 months, at least 18 months, at least 24 months, or at least 36 months); and/or b) a temperature between about 2° C. and about 8° C. for a period of at least 1 month (e.g., at least 3 months, at least 6 months, at least 9 months, at least 12 months, at least 18 months, at least 24 months, or at least 36 months).

In some embodiments, the purity of the crystalline 2-naphthalene sulfonate salt form of Compound (I) (e.g., 2-Napsylate Form I) remains unchanged as measured by HPLC when stored at a temperature of 25° C. for a period of at least 7 days. In some embodiments, the purity of the crystalline 2-naphthalene sulfonate salt form of Compound (I) (e.g., 2-Napsylate Form I) decreases by less than 1% as measured by HPLC when stored at a) a temperature of 25° C. for at least 7 days; b) a temperature of 60° C. for at least 7 days; and/or c) a temperature of 40° C. and a relative humidity of 75% for a period of at least 7 days. In some embodiments, the purity of the crystalline 2-naphthalene sulfonate salt form of Compound (I) (e.g., 2-Napsylate Form I) decreases by less than 0.7% as measured by HPLC when stored at a) a temperature of 25° C. for at least 7 days; b) a temperature of 60° C. for at least 7 days; and/or c) a temperature of 40° C. and a relative humidity of 75% for a period of at least 7 days. In some embodiments, the purity of the 2-naphthalene sulfonate salt form of Compound (I) (e.g., 2-Napsylate Form I) decreases by less than 0.5% as measured by HPLC when stored at a) a temperature of 25° C. for a period of at least 7 days; and/or b) a temperature of 40° C. and a relative humidity of 75% for a period of at least 7 days. In some embodiments, the purity of the 2-naphthalene sulfonate salt form of Compound (I) (e.g., 2-Napsylate Form I) decreases by less than 1% as measured by HPLC when stored at a) a temperature of 25° C. and a relative humidity of 60% for a period of at least 1 month; and/or b) a temperature between 2° C. and 8° C. for a period of at least 1 month. In some embodiments, the purity of the 2-naphthalene sulfonate salt form of Compound (I) (e.g., 2-Napsylate Form I) decreases by 5% or less as measured by HPLC, when stored at a) a temperature of 25° C. and a relative humidity of 60% for a period of at least 1 month (e.g., at least 3 months, at least 6 months, at least 9 months, at least 12 months, at least 18 months, at least 24 months, or at least 36 months); and/or b) a temperature between 2° C. and 8° C. for a period of at least 1 month (e.g., at least 3 months, at least 6 months, at least 9 months, at least 12 months, at least 18 months, at least 24 months, or at least 36 months). In some embodiments, the purity of the 2-naphthalene sulfonate salt form of Compound (I) (e.g., 2-Napsylate Form I) decreases by 3% or less as measured by HPLC, when stored at a) a temperature of about 25° C. and a relative humidity of about 60% for a period of at least 1 month (e.g., at least 3 months, at least 6 months, at least 9 months, at least 12 months, at least 18 months, at least 24 months, or at least 36 months); and/or b) a temperature between about 2° C. and about 8° C. for a period of at least 1 month (e.g., at least 3 months, at least 6 months, at least 9 months, at least 12 months, at least 18 months, at least 24 months, or at least 36 months). In some embodiments, the purity of the 2-naphthalene sulfonate salt form of Compound (I) (e.g., 2-Napsylate Form I) decreases by 1% or less as measured by HPLC, when stored at a) a temperature of about 25° C. and a relative humidity of about 60% for a period of at least 1 month (e.g., at least 3 months, at least 6 months, at least 9 months, at least 12 months, at least 18 months, at least 24 months, or at least 36 months); and/or b) a temperature between about 2° C. and about 8° C. for a period of at least 1 month (e.g., at least 3 months, at least 6 months, at least 9 months, at least 12 months, at least 18 months, at least 24 months, or at least 36 months).

Differential scanning calorimetry (DSC) can be used to assess the physical stability of a solid form of a compound. In some embodiments, the 2-naphthalene sulfonate salt form of Compound (I) melts or decomposes at a temperature greater than about 200° C., for example, greater than about 210° C., greater than about 215° C., greater than about 220° C., greater than about 225° C., or between a temperature of about 200° C. to about 270° C., or between a temperature of about 210° C. to about 260° C., or between a temperature of about 215° C. to about 255° C. In some embodiments, the 2-naphthalene sulfonate salt form of Compound (I), melts or decomposes between a temperature of about 240° C. and about 250° C. In some embodiments, the 2-naphthalene sulfonate salt form of Compound (I) melts or decomposes at a temperature of about 247° C.

In some embodiments, the 2-naphthalene sulfonate salt form of Compound (I) melts or decomposes at a temperature greater than 200° C., for example, greater than 210° C., greater than 215° C., greater than 220° C., greater than 225° C., or between a temperature of 200° C. to 270° C., or between a temperature of 210° C. to 260° C., or between a temperature of 215° C. to 255° C. In some embodiments, the 2-naphthalene sulfonate salt form of Compound (I), melts or decomposes between a temperature of 240° C. and 250° C. In some embodiments, the 2-naphthalene sulfonate salt form of Compound (I) melts or decomposes at a temperature of 247° C.

X-ray powder diffraction (XRPD) is a method that can be used to assess and/or characterize a sample, and can be used to identify indicators of physical instability, such as, e.g., conversion of one crystalline form into another crystalline form and/or to an amorphous form. These indicators of physical instability can be observed by a person of skill in the art using XRPD, for instance, as described in the Examples herein. Conversion of a crystal form to an amorphous form may be observed as either a reduction in the signal to noise ratio (i.e., due to reduced crystallinity) or loss of discrete signals associated with the starting crystalline form in the XRPD pattern, when other parameters such as the sample preparation method are maintained. Conversion to another crystalline form is observed as reduction in signals associated with the starting crystalline form and an increase in signals associated with a different crystalline form. In some embodiments, the crystallinity and/or crystal form of the crystalline 2-naphthalene sulfonate salt form of Compound (I) (e.g., 2-Napsylate Form I) is substantially the same after storage at a) a temperature of 25° C. for at least 7 days; b) a temperature of 60° C. for at least 7 days; and/or c) a temperature of 40° C. and at 75% relative humidity for a period of at least 7 days, as compared to the crystallinity and/or crystal form of the salt form prior to storage, as assessed by XRPD.

In some embodiments, the crystallinity and/or crystal form of the crystalline 2-naphthalene sulfonate salt form of Compound (I) (e.g., 2-Napsylate Form I) is substantially the same after storage at a) a temperature of 25° C. and a relative humidity of 60% for a period of at least 1 month (e.g., at least 3 months, at least 6 months, at least 9 months, at least 12 months, at least 18 months, at least 24 months, or at least 36 months); and/or b) a temperature between 2° C. and 8° C. for a period of at least 1 month (e.g., at least 3 months, at least 6 months, at least 9 months, at least 12 months, at least 18 months, at least 24 months, or at least 36 months), as compared to the crystallinity and/or crystal form of the salt form prior to storage, as assessed by XRPD.

In some embodiments, the crystallinity and/or crystal form of the crystalline 2-naphthalene sulfonate salt form of Compound (I) (e.g., 2-Napsylate Form I) remains unchanged under conditions of variable relative humidity. In some embodiments, the crystallinity and/or crystal form of the crystalline 2-naphthalene sulfonate salt form of Compound (I) (e.g., 2-Napsylate Form I) is substantially the same after the relative humidity is adjusted from 10% to 90% at 25° C. over a period of 36 hours, as compared to the crystallinity and/or crystal form of the salt form prior adjustment of relative humidity. In some embodiments, the crystallinity and/or crystal form of the crystalline 2-naphthalene sulfonate salt form of Compound (I) (e.g., 2-Napsylate Form I) is substantially the same after the relative humidity is cycled from 10% to 90% at 25° C., and back from 90% to 10% at 25° C. over a period of 68 hours, as compared to the crystallinity and/or crystal form of the salt form prior to adjustment of the relative humidity, as assessed by XRPD.

### Methods of Making

In one aspect, this disclosure is directed to a process for preparing a crystalline 2-naphthalene sulfonate salt form of Compound (I), the process comprising:
a) contacting 2-naphthalene sulfonic acid with a solution comprising Compound (I) to form a mixture comprising a crystalline 2-naphthalene sulfonate salt form of Compound (I), wherein the solution comprising Compound (I) has been prepared in a suitable solvent; and
b) isolating the crystalline 2-naphthalene sulfonate salt form of Compound (I) from the mixture.

In some embodiments, a suitable solvent useful for preparing a solution comprising Compound (I) is a pro-solvent (i.e., a solvent Compound (I) is soluble in). In some embodiments, the suitable solvent is 2-methyltetrahydrofuran, tetrahydrofuran, ethyl acetate, an alcohol (e.g., isopropyl alcohol, ethanol, methanol, n-butanol, and the like), dimethoxyethane, or a combination of two or more thereof. In some embodiments, the solvent is an alcohol. In some embodiments, the solvent is n-butanol.

In some embodiments, 2-naphthalene sulfonic acid is added directly to the solution comprising Compound (I) to form a mixture, for example a slurry. In other embodiments, 2-naphthalene sulfonic acid is first dissolved in a suitable solvent, and next added to the solution comprising Compound (I). In some embodiments, the suitable solvent is a pro-solvent (i.e., a solvent 2-naphthalene sulfonic acid is soluble in). In some embodiments, 2-naphthalene sulfonic acid is dissolved in one or more of the solvents used in step a). In some embodiments, 2-naphthalene sulfonic acid is dissolved in a solvent that was not used in step a). In some embodiments, 2-naphthalene sulfonic acid is first dissolved in an alcohol and next added to the solution comprising Compound (I) to form a slurry. In some embodiments, 2-naphthalene sulfonic acid is first dissolved in an ether (e.g., tetrahydrofuran, 2-methyltetrahydrofuran, diethyl ether, and the like) and next added to the solution comprising Compound (I) to form a slurry. In some embodiments, 2-naphthalene sulfonic acid is first dissolved in 2-methyltetrahydrofuran and next added to the solution comprising Compound (I) to form a slurry.

In some embodiments, the method comprises:
contacting a solution of 2-naphthalene sulfonic acid in 2-methyltetrahydrofuran to a solution of Compound (I) in n-butanol to form a slurry; and
isolating the solids from the slurry to obtain the crystalline 2-naphthalene sulfonate salt form of Compound (I).

The solution of 2-naphthalene sulfonic acid in a suitable solvent can be added to the solution comprising Compound (I) rapidly, e.g., by pouring the entire solution at once, or the 2-naphthalene sulfonic acid solution can be added slowly over time. In some embodiments, a first portion of the 2-naphthalene sulfonic acid solution can be added to the solution comprising Compound (I) initially, while the remainder of the 2-naphthalene sulfonic acid solution is added slowly over time. In some embodiments, the 2-naphthalene sulfonic acid solution is added to the solution comprising Compound (I) slowly over time. Slow addition can be done by, for example, adding the solution drop-wise or portion-wise, or the solution can be added continuously over time using a syringe pump.

In some embodiments, the volume of the 2-naphthalene sulfonic acid solution that is added to the solution comprising Compound (I) is less than the starting volume of the solution comprising Compound (I). In some embodiments, the volume ratio of the solution comprising Compound (I) to the 2-naphthalene sulfonic acid solution is 2:1, 3:1, 4:1, 5:1, or 6:1.

In some embodiments, the stoichiometric ratio of the 2-naphthalene sulfonic acid to Compound (I) is about 1:1. In some embodiments, the stoichiometric ratio of the 2-naphthalene sulfonic acid to Compound (I) is less than 1:1. In some embodiments, the stoichiometric ratio of the 2-naphthalene sulfonic acid to Compound (I) is 0.75:1, 0.80:1, 0.85:1, 0.90:1, 0.95:1, or 1:1. In some embodiments, the stoichiometric ratio of the 2-naphthalene sulfonic acid to Compound (I) is between 0.90:1 and 1:1. In some embodiments, the stoichiometric ratio of the 2-naphthalene sulfonic acid to Compound (I) is between 0.95:1 and 1:1.

The slurry formed from adding the 2-naphthalene sulfonic acid to the solution comprising Compound (I) can be allowed to stir over a period of time prior to isolating the solids from the slurry. In some embodiments, the slurry is allowed to stir at an elevated temperature. In some embodiments, the slurry is allowed to stir at a temperature between 30° C. and 80° C., e.g., 35° C., 40° C., 45° C., 50° C., 55° C., 60° C., 65° C., 70° C. or 75° C. In some embodiments, the slurry is subsequently cooled from the elevated temperature prior to isolating the solids from the slurry.

Several methods, including filtration, decantation, and solvent evaporation can be used to isolate the solids from the slurry to obtain the crystalline 2-naphthalene sulfonate salt form of Compound (I). In some embodiments, the solids are isolated from the slurry using vacuum filtration. The isolated solids can optionally be washed with an anti-solvent, e.g., 2-methylthetrahydrofuran, n-butanol, or mixtures thereof. Washing the solids with an anti-solvent can be accomplished via resuspending the solids in the anti-solvent and re-isolating using the methods described above, or by pouring the anti-solvent over the solids on a filter. The isolated solids can then be further air-dried, dried under vacuum, and/or dried under a stream of humid nitrogen to afford the crystalline 2-naphthalene sulfonate salt form of Compound (I).

### Pharmaceutical Compositions

The 2-naphthalene sulfonate salt forms of Compound (I) of the present disclosure may be in the form of compositions suitable for administration to a subject. The disclosure provides a pharmaceutical composition comprising a compound according to this disclosure and one or more pharmaceutically acceptable excipients. More particularly, the disclosure provides a pharmaceutical composition comprising a 2-naphthalene sulfonate salt and one or more pharmaceutically acceptable excipients. In certain embodiments, the 2-naphthalene sulfonate salt form of Compound (I) may be present in an effective amount. The pharmaceutical compositions may be used in the methods of the present disclosure; thus, for example, the pharmaceutical compositions comprising a compound according to this disclosure can be administered to a subject in order to practice the therapeutic and prophylactic methods and uses described herein.

In one or more embodiments, the pharmaceutical composition comprises the 2-naphthalene sulfonate salt form of Compound (I) in an amount that achieves a freebase equivalent of between about 10 mg to about 1,000 mg. In one or more embodiments, the pharmaceutical composition comprises the 2-naphthalene sulfonate salt form of Compound (I) in an amount that achieves a freebase equivalent of between about 10 mg to about 500 mg. In some embodiments, the 2-naphthalene sulfonate salt form of Compound (I) (e.g., 2-Napsylate Form I) is present in an amount that achieves a freebase equivalent of between about 10 mg to about 300 mg, such as, for example, 10 mg, 15 mg, 20 mg, 25 mg, 30 mg, 35, mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, or 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 500 mg. In some embodiments, the 2-naphthalene sulfonate salt form of Compound (I) is present in an amount that achieves a freebase equivalent of between about 10 mg and about 50 mg. In some embodiments, the 2-naphthalene sulfonate salt form of Compound (I) is present in an amount that achieves a freebase equivalent of between about 25 mg to about 75 mg. In some embodiments, the 2-naphthalene sulfonate salt form of Compound (I) is present in an amount that achieves a freebase equivalent of between about 25 mg to about 50 mg. In some embodiments, the 2-naphthalene sulfonate salt form of Compound (I) is present in an amount that achieves a freebase equivalent of about 40 mg. In some embodiments, the 2-naphthalene sulfonate salt form of Compound (I) is present in an amount that achieves a freebase equivalent of between about 50 mg to about 100 mg. In some embodiments, the 2-naphthalene sulfonate salt form of Compound (I) is present in an amount that achieves a freebase equivalent of about 80 mg. In some embodiments, the 2-naphthalene sulfonate salt form of Compound (I) is present in an amount that achieves a freebase equivalent of about 100 mg. In some embodiments, the 2-naphthalene sulfonate salt form of Compound (I) is present in an amount that achieves a freebase equivalent of between about 100 mg to about 200 mg. In some embodiments, the 2-naphthalene sulfonate salt form of Compound (I) is present in an amount that achieves a freebase equivalent of between about 100 mg to about 150 mg. In some embodiments, the 2-naphthalene sulfonate salt form of Compound (I) is present in an amount that achieves a freebase equivalent of about 120 mg. In some embodiments, the 2-naphthalene sulfonate salt form of Compound (I) is present in an amount that achieves a freebase equivalent of between about 150 mg to about 200 mg. In some embodiments, the 2-naphthalene sulfonate salt form of Compound (I) is present in an amount that achieves a freebase equivalent of between about 200 mg to about 300 mg. In some embodiments, the 2-naphthalene sulfonate salt form of Compound (I) is present in an amount that achieves a freebase equivalent of between about 200 mg to about 250 mg. In some embodiments, the 2-naphthalene sulfonate salt form of Compound (I) is present in an amount that achieves a freebase equivalent of between about 250 mg to about 300 mg. In some embodiments, the 2-naphthalene sulfonate salt form of Compound (I) is present in an amount that achieves a freebase equivalent of between about 300 mg to about 400 mg. In some embodiments, the 2-naphthalene sulfonate salt form of Compound (I) is present in an amount that achieves a freebase equivalent of between about 300 mg to about 350 mg. In some embodiments, the 2-naphthalene sulfonate salt form of Compound (I) is present in an amount that achieves a freebase equivalent of between about 350 mg to about 400 mg. In some embodiments, the 2-naphthalene sulfonate salt form of Compound (I) is present in an amount that achieves a freebase equivalent of between about 400 mg to about 500 mg. In some embodiments, the 2-naphthalene sulfonate salt form of Compound (I) is present in an amount that achieves a freebase equivalent of between about 400 mg to about 450 mg. In some embodiments, the 2-naphthalene sulfonate salt form of Compound (I) is 2-Napsylate Form I and is present in an amount that achieves a freebase equivalent of between about 450 mg to about 500 mg.

The pharmaceutical compositions of the present disclosure can be formulated to be compatible with the intended method or route of administration. Routes of administration may include those known in the art. Exemplary routes of administration are oral and parenteral. Furthermore, the pharmaceutical compositions may be used in combination with one or more other therapies described herein in order to treat or prevent the diseases, disorders and conditions as contemplated by the present disclosure. In one embodiment, one or more other therapeutic agents contemplated by this disclosure are included in the same pharmaceutical composition that comprises a 2-naphthalene sulfonate salt form of Compound (I). In another embodiment, the one or more other therapeutical agents are in a composition that is separate from the pharmaceutical composition comprising the 2-naphthalene sulfonate salt form of Compound (I) (e.g., 2-Napsylate Form I).

In one aspect, the compounds described herein may be administered orally. Oral administration may be via, for example, capsules or tablets. In making the pharmaceutical compositions that include the 2-naphthalene sulfonate salt form of Compound (I) (e.g., 2-Napsylate Form I), the tablet or capsule typically includes at least one pharmaceutically acceptable excipient.

Non-limiting examples of pharmaceutically acceptable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, sterile water, syrup, and methyl cellulose. Additional pharmaceutically acceptable excipients include lubricating agents such as talc, magnesium stearate, and mineral oil; wetting agents; emulsifying and suspending agents; preserving agents such as methyl and propylhydroxy-benzoates.

In another aspect, the compounds described herein may be administered parenterally, for example by intravenous injection. A pharmaceutical composition appropriate for parenteral administration may be formulated in solution for injection or may be reconstituted for injection in an appropriate system such as a physiological solution. Such solutions may include sterile water for injection, salts, buffers, and tonicity excipients in amounts appropriate to achieve isotonicity with the appropriate physiology.

The pharmaceutical compositions described herein may be stored in an appropriate sterile container or containers. In some embodiments, the container is designed to maintain stability for the pharmaceutical composition over a given period of time.

### Administering

In general, the disclosed methods comprise administering a 2-naphthalene sulfonate salt form of Compound (I) (e.g., 2-Napsylate Form I), or a composition thereof, in an effective amount to a subject in need thereof. An “effective amount” with reference to a 2-naphthalene sulfonate salt form of Compound (I) (e.g., 2-Napsylate Form I) according to the present disclosure means an amount of the compound that is sufficient to engage the target (i.e., AXL) (by inhibiting or antagonizing the target) at a level that is indicative of the potency of the compound. For AXL, target engagement can be determined by one or more biochemical or cellular assays resulting in an IC50, or similar value which can be used as one assessment of the potency of the compound. The effective amount may be administered as a single quantity or as multiple, smaller quantities (e.g., as one tablet with “x” amount, as two tablets each with “x/2” amount, etc.).

In some embodiments, the disclosed methods comprise administering a therapeutically effective amount of a 2-naphthalene sulfonate salt form of Compound (I) (e.g., 2-Napsylate Form I) according to this disclosure to a subject in need thereof. As used herein, the phrase “therapeutically effective amount” with reference to AXL means a dose regimen (i.e., amount and interval) of the compound that provides the specific pharmacological effect for which the compound is administered to a subject in need of such treatment. For prophylactic use, a therapeutically effective amount may be effective to eliminate or reduce the risk, lessen the severity, or delay the onset of the disease, including biochemical, histological and/or behavioral signs or symptoms of the disease. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. With respect to cancer specifically, a therapeutically effective amount may, for example, result in the killing of cancer cells, reduce cancer cell counts, reduce tumor burden, reduce tumor volume, eliminate tumors or metastasis, or reduce metastatic spread. A therapeutically effective amount may vary based on, for example, one or more of the following: the age and weight of the subject, the subject's overall health, the stage of the subject's disease, the route of administration, and prior or concomitant treatments.

Administration may comprise one or more (e.g., one, two, or three or more) dosing cycles.

In certain embodiments, the 2-naphthalene sulfonate salt form of Compound (I) contemplated by the present disclosure may be administered (e.g., orally, parenterally, etc.) at about 0.01 mg/kg to about 100 mg/kg, or about 0.1 mg/kg to about 50 mg/kg, or about 0.1 mg/kg to about 25 mg/kg, or about 0.1 mg/kg to about 15 mg/kg, or about 0.1 mg/kg to about 10 mg/kg, or about 0.1 mg/kg to about 5 mg/kg of subject's body weight per day, one or more times a day, a week, or a month, to obtain the desired effect. In some embodiments, a suitable weight-based dose of a compound contemplated by the present disclosure is used to determine a dose that is administered independent of a subject's body weight. In certain embodiments, the 2-naphthalene sulfonate salt form of Compound (I) of the present disclosure is administered (e.g., orally, parenterally, etc.) at fixed dosage levels that achieve freebase equivalents of about 1 mg to about 1000 mg one or more times a day, a week, or a month, to obtain the desired effect. In some embodiments, the 2-naphthalene sulfonate salt form of Compound (I) according to the present disclosure is administered at a fixed dosage level that achieves freebase equivalents of between about 10 mg to about 500 mg one or more times a day, a week, or a month, to obtain the desired effect. In some embodiments, the 2-naphthalene sulfonate salt form of Compound (I) according to the present disclosure is administered at a fixed dosage level that achieves freebase equivalents of between about 10 mg to about 300 mg, particularly 10 mg, 15 mg, 20 mg, 25 mg, 30 mg, 35, mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, or 300 mg, one or more times a day, a week, or a month, to obtain the desired effect. In some embodiments, the 2-naphthalene sulfonate salt form of Compound (I) (e.g., 2-Napsylate Form I) according to the present disclosure is administered in an amount that achieves freebase equivalents of between about 10 mg and about 50 mg one or more times a day, a week, or a month, to obtain the desired effect. In some embodiments, the 2-naphthalene sulfonate salt form of Compound (I) (e.g., 2-Napsylate Form I) according to the present disclosure is administered in an amount that achieves freebase equivalents of between about 25 mg to about 75 mg one or more times a day, a week, or a month, to obtain the desired effect. In some embodiments, the 2-naphthalene sulfonate salt form of Compound (I) (e.g., 2-Napsylate Form I) according to the present disclosure is administered in an amount that achieves freebase equivalents of between about 50 mg to about 100 mg one or more times a day, a week, or a month, to obtain the desired effect. In some embodiments, the 2-naphthalene sulfonate salt form of Compound (I) (e.g., 2-Napsylate Form I) according to the present disclosure is administered in an amount that achieves freebase equivalents of between about 100 mg to about 200 mg one or more times a day, a week, or a month, to obtain the desired effect. In some embodiments, the 2-naphthalene sulfonate salt form of Compound (I) (e.g., 2-Napsylate Form I) according to the present disclosure is administered in an amount that achieves freebase equivalents of between about 100 mg to about 150 mg one or more times a day, a week, or a month, to obtain the desired effect. In some embodiments, the 2-naphthalene sulfonate salt form of Compound (I) (e.g., 2-Napsylate Form I) according to the present disclosure is administered in an amount that achieves freebase equivalents of between about 200 mg to about 300 mg one or more times a day, a week, or a month, to obtain the desired effect. In some embodiments, the 2-naphthalene sulfonate salt form of Compound (I) (e.g., 2-Napsylate Form I) according to the present disclosure is administered in an amount that achieves freebase equivalents of between about 250 mg to about 300 mg one or more times a day, a week, or a month, to obtain the desired effect.

In some embodiments, the 2-naphthalene sulfonate salt form of Compound (I) (e.g., 2-Napsylate Form I) according to the present disclosure is administered one or more times a day, a week, or a month to obtain a desired effect. In some embodiments, the 2-naphthalene sulfonate salt form of Compound (I) (e.g., 2-Napsylate Form I) is administered once or twice a day. In one embodiment, the 2-naphthalene sulfonate salt form of Compound (I) (e.g., 2-Napsylate Form I) is administered twice daily. In another embodiment, the 2-naphthalene sulfonate salt form of Compound (I) (e.g., 2-Napsylate Form I) is administered once daily.

In certain embodiments, the 2-naphthalene sulfonate salt form of Compound (I) (e.g., 2-Napsylate Form I) is contained in a “unit dosage form”. The phrase “unit dosage form” refers to physically discrete units, each unit containing a predetermined amount of the 2-naphthalene sulfonate salt form of Compound (I) (e.g., 2-Napsylate Form I), either alone or in combination with one or more additional agents, sufficient to produce the desired effect. It will be appreciated that the parameters of a unit dosage form will depend on the particular agent and the effect to be achieved.

### Methods of Use

The present disclosure provides methods for using the 2-naphthalene sulfonate salt forms of Compound (I) described herein in the preparation of a medicament for inhibition of AXL. As used herein, the terms “inhibit”, ‘inhibition” and the like refer to the ability of an antagonist to decrease the function or activity of a particular target, e.g., AXL. The decrease is preferably at least a 50% and may be, for example, at least about 55%, at least about 60%, at least about 65%, at least about 70%, at least about 75%, at least about 80%, at least about 85%, at least about 90%, or at least about 95%. The present disclosure also encompasses the use of the 2-naphthalene sulfonate salt forms of Compound (I) described herein in the preparation of a medicament for the treatment or prevention of diseases, disorders, and/or conditions that would benefit from inhibition of AXL. As one example, the present disclosure encompasses the use of the compounds described herein in the preparation of a medicament for the treatment of cancer. In some embodiments of the aforementioned methods, the compounds described herein are used in combination with at least one additional therapy, examples of which are set forth elsewhere herein.

### Oncology and Oncology-Related Disorders.

In one or more embodiments, the 2-naphthalene sulfonate salt forms of Compound (I) described herein (e.g., 2-Napsylate Form I) are useful in the treatment and/or prophylaxis of cancer (e.g., carcinomas, sarcomas, leukemias, lymphomas, myelomas, etc.). In certain embodiments, the cancer may be locally advanced and/or unresectable, metastatic, or at risk of becoming metastatic. Alternatively, or in addition, the cancer may be recurrent or no longer responding to a treatment, such as a standard of care treatment known to one of skill in the art. Exemplary types of cancer contemplated by this disclosure include cancer of the genitourinary tract (e.g., bladder, kidney, renal cell, penile, prostate, testicular, ovary, cervix, uterus, Von Hippel-Lindau disease, etc.), breast, gastrointestinal tract (e.g., esophagus, oropharynx, stomach, small or large intestines, colon, or rectum), bone, bone marrow, skin (e.g., melanoma, squamous cell carcinoma, or basal cell carcinoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), and the immune system (e.g., spleen or thymus). In some embodiments, the patient having cancer is determined to have an STK11 mutation.

In some embodiments, the 2-naphthalene sulfonate salt forms of Compound (I) described herein (e.g., 2-Napsylate Form I) are useful in the treatment and/or prophylaxis of hematological malignancies. Exemplary types of cancer affecting the hematopoietic system include leukemias, lymphomas and myelomas, including acute myeloid leukemia, adult T-cell leukemia, T-cell large granular lymphocyte leukemia, acute lymphoblastic leukemia, chronic lymphocytic leukemia, chronic myelogenous leukemia, acute monocytic leukemia, Hodgkin's and Non-Hodgkin's lymphoma, Diffuse large B Cell lymphoma, and multiple myeloma. In some embodiments, the hematological malignancy is acute myeloid leukemia.

In another embodiment, the 2-naphthalene sulfonate salt forms of Compound (I) described herein (e.g., 2-Napsylate Form I) are useful in the treatment and/or prophylaxis of solid tumors. The solid tumor may be, for example, ovarian cancer, fallopian tube cancer, primary peritoneal cancer, endometrial cancer, breast cancer, lung cancer (small cell or non-small cell), colon cancer, prostate cancer, cervical cancer, biliary cancer, pancreatic cancer, gastric cancer, esophageal cancer, liver cancer (hepatocellular carcinoma), kidney cancer (renal cell carcinoma), head-and-neck tumors, mesothelioma, melanoma, sarcomas, central nervous system (CNS) hemangioblastomas, and brain tumors (e.g., gliomas, such as astrocytoma, oligodendroglioma and glioblastomas). In some embodiments, the solid tumor is lung cancer (NSCLC), colorectal cancer (CRC), breast cancer, ovarian cancer, kidney cancer (renal cell carcinoma, RCC), head and neck cancer (HNSCC), or bladder cancer.

In some embodiments, the 2-naphthalene sulfonate salt forms of Compound (I) described herein (e.g., 2-Napsylate Form I) are useful in the treatment lung cancer (e.g., non-small cell lung cancer (NSCLC)), pancreatic cancer (e.g., pancreatic ductal adenocarcinoma (PDAC)), ovarian cancer (e.g., epithelial ovarian cancer (EOC), high grade serous ovarian cancer (HSOC), or platinum resistant ovarian cancer (PROC)), breast cancer (e.g., triple negative breast cancer (TNBC)), bladder cancer (including urothelial malignancies of the renal pelvis and ureter), colorectal cancer (CRC), head and neck cancer (e.g., head and neck squamous cell carcinoma (HNSCC)), kidney cancer (e.g., renal cell carcinoma (RCC) or clear cell renal cell carcinoma (ccRCC)), liver cancer, glioblastoma, mesothelioma, melanoma, or leukemia (e.g., acute myeloid leukemia (AML), or myelodysplastic syndrome). In some embodiments, the cancer is lung cancer, pancreatic cancer, ovarian cancer, breast cancer, head and neck cancer, kidney cancer, leukemia, or myelodysplastic syndrome. In one embodiment, the cancer is NSCLC, PDAC, ccRCC, or AML.

In some embodiments, the 2-naphthalene sulfonate salt forms of Compound (I) described herein (e.g., 2-Napsylate Form I) are useful in the treatment of kidney cancer. In further embodiments, the kidney cancer is renal cell carcinoma. In still further embodiments, the renal cell carcinoma is clear cell renal carcinoma (ccRCC).

In some embodiments, the 2-naphthalene sulfonate salt forms of Compound (I) described herein (e.g., 2-Napsylate Form I) are useful in the treatment of lung cancer. In further embodiments, the lung cancer is non-small cell lung cancer (NSCLC). In still further embodiments, the NSCLC is lung squamous cell carcinoma or lung adenocarcinoma. In some embodiments the NSCLC is EGFR mutant NSCLC. In some embodiments, the lung cancer is squamous cell carcinoma (SCC). In some embodiments, the NSCLC is STK11 mutant NSCLC. In some embodiments, the lung cancer is non-squamous NSCLC with documented mutation or deletion in the STK11 gene. In some embodiments, the NSCLC has relapsed to therapeutics, including, but not limited to, anti-PD-1, anti-PD-L1, or other immunotherapies, targeted therapy, or chemotherapy (e.g., platinum containing chemotherapy). In some embodiments, the cancer is negative for mutations of EGFR, ALK, ROS1, NTRK, C-MET, or RET.

In some embodiments, the 2-naphthalene sulfonate salt forms of Compound (I) described herein (e.g., 2-Napsylate Form I) are useful in the treatment of head and neck cancer. In further embodiments, the head and neck cancer is head and neck squamous cell carcinoma (HNSCC). In some embodiments, the HNSCC is relapsed or resistant to radiation, chemotherapy, or immunotherapy.

In some embodiments, the 2-naphthalene sulfonate salt forms of Compound (I) described herein (e.g., 2-Napsylate Form I) are useful in the treatment of leukemia or myelodysplastic syndrome (MDS). In further embodiments, the leukemia is acute myeloid leukemia (AML). In still further embodiments the AML or MDS is relapsed or refractory AML or MDS. In some embodiments, the AML is FLT-3 mutant AML.

In some embodiments, the 2-naphthalene sulfonate salt forms of Compound (I) described herein (e.g., 2-Napsylate Form I) are useful in the treatment of breast cancer. In further embodiments, the breast cancer is hormone receptor positive (e.g., ERa-positive breast cancer, PR-positive breast cancer, ERa-positive and PR-positive breast cancer), HER2 positive breast cancer, HER2 over-expressing breast cancer, or any combination thereof. In still further embodiments, the breast cancer is triple negative breast cancer (TNBC).

In some embodiments, the 2-naphthalene sulfonate salt forms of Compound (I) described herein (e.g., 2-Napsylate Form I) are useful in the treatment of pancreatic cancer. In further embodiments, the pancreatic cancer is pancreatic neuroendocrine tumor (PNET) or pancreatic adenocarcinoma (i.e., pancreatic ductal adenocarcinoma (PDAC)).

In some embodiments, the 2-naphthalene sulfonate salt forms of Compound (I) described herein (e.g., 2-Napsylate Form I) are useful in the treatment of ovarian cancer. In further embodiments the ovarian cancer is metastatic. In further embodiments, the ovarian cancer is epithelial ovarian cancer (EOC). In some embodiments, the ovarian cancer is high grade serous ovarian cancer (HGSOC). In some embodiments, the ovarian cancer is characterized by a mesenchymal (MES) molecular subtype. In some embodiments, the ovarian cancer is resistant to therapy, including but not limited to platinum-based or taxane-based therapies (e.g., platinum-resistant ovarian cancer (PROC)).

In one or more embodiments, the cancer is an oncogene addicted cancer. Oncogene addicted cancers are those that rely on a dominant oncogene for growth and survival, such as, for example, ALK, ABL, AURORA, AKT, PDGFR, KIT, EGFR, VEGFR, FGFR3, FLT-3, MYC, RET, BRAF, PI3K, NF-κB, JAK, STAT, BCL-2, MCL-1, KRAS, HRAS, MEK, ERK, HER-2, HER-3 or MET. In one embodiment, the oncogene is KRAS.

In the aforementioned embodiments, the methods of the present disclosure may be practiced in an adjuvant setting or neoadjuvant setting. The methods described herein may be indicated as a first line, second line, third line, or greater line of treatment. In some embodiments, the subject has been treated with one or more prior lines of therapy. In some embodiments, the prior line of therapy included a platinum-based chemotherapeutic agent and/or an immune checkpoint inhibitor (e.g., a PD-1 or PD-L1 inhibitor).

In some embodiments, the methods of the present disclosure may be indicated as a first line therapy in subjects identified as having a deletion or mutation of the STK11 gene. In some embodiments, the methods of the present disclosure may be indicated as a second line therapy in subjects identified as having a deletion or mutation of the STK11 gene. In another embodiment, the methods of the present disclosure may be indicated as a second line therapy in subjects identified as having a cancer resistant to therapy (e.g., resistance to chemotherapy, immunotherapy, radiation, etc.). In one embodiment, standard therapy is ineffective, intolerable, or considered inappropriate for treatment of the patient's cancer.

In general, the disclosed methods for treating or preventing cancer, or a cancer-related disease, disorder or condition, in a subject in need thereof comprise administering to the subject a compound disclosed here. In some embodiments, the present disclosure provides methods for treating or preventing cancer, or a cancer-related disease, disorder or condition with a compound disclosed herein and at least one additional therapy, examples of which are set forth elsewhere herein.

### Combination Therapy

The present disclosure contemplates the use of the 2-naphthalene sulfonate salt forms of Compound (I) described herein alone or in combination with one or more additional therapy. Each additional therapy can be a therapeutic agent or another treatment modality. In embodiments comprising one or more additional therapeutic agents, each agent may target a different, but complementary, mechanism of action. The use of the compounds of this disclosure in combination with one or more additional therapies may have a synergistic therapeutic or prophylactic effect on the underlying disease, disorder, or condition. In addition or alternatively, the combination therapy may allow for a dose reduction of one or more of the therapies, thereby ameliorating, reducing or eliminating adverse effects associated with one or more of the agents.

In embodiments comprising one or more additional treatment modality, the 2-naphthalene sulfonate salt forms of Compound (I) described herein (e.g., 2-Napsylate Form I) can be administered before, after or during treatment with the additional treatment modality. In embodiments comprising one or more additional therapeutic agent, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

### Cancer Therapies

The present disclosure contemplates the use of the 2-naphthalene sulfonate salt forms of Compound (I) described herein (e.g., 2-Napsylate Form I) in combination with one or more additional therapies useful in the treatment of cancer.

In some embodiments, one or more of the additional therapies is an additional treatment modality. Exemplary treatment modalities include but are not limited to surgical resection of a tumor, bone marrow transplant, radiation therapy, and photodynamic therapy.

In some embodiments, one or more of the additional therapies is a therapeutic agent. Exemplary therapeutic agents include chemotherapeutic agents, radiopharmaceuticals, hormone therapies, epigenetic modulators, ATP-adenosine axis-targeting agents, targeted therapies, signal transduction inhibitors, RAS signaling inhibitors, PI3K inhibitors, arginase inhibitors, HIF inhibitors, PAK4 inhibitors, immunotherapeutic agents, cellular therapies, gene therapies, immune checkpoint inhibitors, and agonists of stimulatory or co-stimulatory immune checkpoints. In one or more embodiments, one or more of the additional therapies is selected from the groups consisting of inhibitors of CD47-SIRPa pathway, kinase inhibitors, inhibitors of HIF, inhibitors of PARP, RAS signaling inhibitors, immune checkpoint inhibitors, agents that target the extracellular production of adenosine, radiation therapy, and chemotherapeutic agents. In some embodiments, the one or more additional therapies comprise a kinase inhibitor. In some embodiments, the one or more additional therapies comprise an immune checkpoint inhibitor and/or a chemotherapeutic agent.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; proteasome inhibitors such as bortezomib, carfilzomib and ixazomib; topoisomerase inhibitors such as irinotecan, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above. In certain embodiments, combination therapy comprises a chemotherapy regimen that includes one or more chemotherapeutic agents. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), CAPOX (capecitabine and oxaliplatin), a taxoid (e.g., docetaxel, paclitaxel, nab-paclitaxel,etc.), a fluoropyrimidine-containing chemotherapeutic agent (e.g., fluorouracil, capecitabine, floxuridine), a platinum containing chemotherapeutic agent, low-dose cytarabine (LDAC), and/or gemcitabine. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), a taxoid (e.g., docetaxel, paclitaxel, nab-paclitaxel, etc.), low-dose cytarabine (LDAC), gemcitabine, and/or platinum based therapies (e.g., cisplatin, carboplatin, and oxaliplatin).

In some embodiments, the chemotherapeutic agent comprises gemcitabine, cytarabine, or a platinum-based, taxoid-based, or anthracycline-based chemotherapeutic agent.

In some embodiments, the chemotherapeutic agent is selected from the group consisting of cisplatin, carboplatin, oxaliplatin, doxorubicin, docetaxel, nab-paclitaxel, and paclitaxel.

In some embodiments, the chemotherapeutic agent comprises of cisplatin, carboplatin, or oxaliplatin.

In some embodiments, the chemotherapeutic agent comprises docetaxel, nab-paclitaxel, or paclitaxel.

In some embodiments, one or more of the additional therapeutic agents is a signal transduction inhibitor. Signal transduction inhibitors are agents that selectively inhibit one or more steps in a signaling pathway. Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., CLN-081, gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab) and HER-3 receptor inhibitors; (iv) vascular endothelial growth factor receptor (VEGFR) inhibitors including small molecule inhibitors (e.g., axitinib, regorafenib, sunitinib and sorafenib), VEGF kinase inhibitors (e.g., lenvatinib, cabozantinib, pazopanib, tivozanib, XL092, etc.), anti-VEGF antibodies (e.g., bevacizumab), and anti-VEGFR antibodies (e.g., ramucirumab); (v) inhibitors of AKT family kinases or the AKT pathway (e.g., rapamycin); (vi) inhibitors of mTOR, such as, for example, everolimus, sirolimus, temsirolimus; (vii) inhibitors of serine/threonine-protein kinase B-Raf (BRAF), such as, for example, vemurafenib, dabrafenib and encorafenib; (viii) inhibitors of rearranged during transfection (RET), including, for example, selpercatinib and pralsetinib; (ix) tyrosine-protein kinase Met (MET) inhibitors (e.g., tepotinib, tivantinib, cabozantinib and crizotinib); (x) anaplastic lymphoma kinase (ALK) inhibitors (e.g., ensartinib, ceritinib, lorlatinib, crizotinib, and brigatinib); (xi) inhibitors of the RAS signaling pathway (e.g., inhibitors of KRAS, HRAS, RAF, MEK, ERK) as described elsewhere herein; (xii) FLT-3 inhibitors (e.g., gilteritinib); (xiii) inhibitors of Trop-2; (xiv) inhibitors of the JAK/STAT pathway, e.g., JAK inhibitors including tofacitinib and ruxolitinib, or STAT inhibitors such as napabucasin; (xv) inhibitors of NF-wB; (xvi) cell cycle kinase inhibitors (e.g., flavopiridol); (xvii) phosphatidyl inositol kinase (PI3K) inhibitors; (xviii) protein kinase B (AKT) inhibitors (e.g., capivasertib, miransertib); (xix) platelet-derived growth factor receptor (PDGFR) inhibitors (e.g., imatinib, sunitinib, regorafenib, avapritinib, lenvatinib, nintedanib, famitinib, ponatinib, axitinib, repretinib, etc.); (xx) insulin-like growth factor receptor (IGFR) inhibitors (e.g., erlotinib, afatinib, gefitinib, osimertinib, dacomitinib); (xxi) fibroblast growth factor receptor (FGFR) inhibitors (e.g., futibatinib, erdafitinib, pemigatinib); and (xxii) receptor tyrosine kinase KIT inhibitors (e.g., imatinib, sorafenib, sunitinib, masitinib, repretinib, avapritinib). In one or more embodiments, the one or more additional therapeutic agents comprise an inhibitor of EGFR, VEGFR, PDGFR, IGFR, HER-2, HER-3, BRAF, RET, MET, ALK, RAS (e.g., KRAS, MEK, ERK), FLT-3, JAK, STAT, NF-κB, PI3K, and/or AKT, or any combinations thereof. In some embodiments, the one or more additional therapeutic agents comprise one or more kinase inhibitors that inhibit of one or more of EGFR, VEGFR, HER-2, HER-3, BRAF, PDGFR, c-MET, MEK, ERK, ALK, RET, KIT, IGFR, TRK, and/or FGFR. In some embodiments, the one or more additional therapeutic agents comprise one or more kinase inhibitors that inhibit one or more of EGFR, VEGFR, and/or c-MET. In some embodiments, the one or more additional therapeutic agents comprise one or more kinase inhibitors that inhibito one or more of EGFR, KIT, PDGFR, and/or BRAF. In some embodiments, the one or more additional therapeutic agents comprise one or more kinase inhibitors that inhibit one or more of EDGFR, ERK, BRAF, and/or KIT.

In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963, everolimus. Direct inhibitors of RAS mutants have also been explored, and generally target the KRAS-GTP complex or the KRAS-GDP complex. Exemplary direct RAS inhibitors under development include, but are not limited to, sotorasib (AMG510), adagrasib (MRTX849), mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments, one or more of the additional therapeutic agents is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present invention contemplates the use of the 2-naphthalene sulfonate salt forms of Compound (I) described herein (e.g., 2-Napsylate Form I) in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CPI” may be used herein interchangeably. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (Tcell immunoglobulin and mucin domain containing protein 3); LAG-3 (lymphocyte activation gene 3); TIGIT (T cell immunoreceptor with Ig and ITIM domains); CD276 (B7-H3), PD-L2, Galectin 9, CEACAM-1, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii) C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). In some embodiments, the checkpoint inhibitor inhibits one or more of PD-1, PD-L1, BTLA, LAG-3, a B7 family member, TIM-3, TIGIT or CTLA-4.

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MEDI5752 and KN046.

In some embodiments, an immune checkpoint inhibitor is a PD-1 antagonist. In further embodiments, the PD-1 antagonist can be an antagonistic PD-1 antibody, small molecule or peptide. Suitable antagonistic PD-1 antibodies include, for example, monospecific antibodies such as balstilimab, budigalimab, camrelizumab, cetrelimab, cosibelimab, dostarlimab, cemiplimab, ezabenlimab, MEDI-0680 (AMP-514; WO2012/145493), nivolumab, pembrolizumab, penpulimab, pidilizumab, pimivalimab, retifanlimab, sasanlimab, serplulimab, spartalizumab, sintilmab, tislelizumab, toripalimab, and zimberelimab; as well as bi-specific antibodies such as LY3434172, IBI321, ivonescimab, rilvegostomig, tebotelimab, and tobemstomig. In still further embodiments, the PD-1 antagonist can be a recombinant protein composed of the extracellular domain of PD-L2 (B7-DC) fused to the Fc portion of IgGl (AMP-224). In certain embodiments, an immune checkpoint inhibitor is zimberelimab.

In some embodiments, the immune checkpoint inhibitor is a PD-L1 antagonist. In further embodiments, the PD-L1 antagonist can be an antagonistic PD-L1 antibody. Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist. In further embodiments, the TIGIT antagonist can be an antagonistic TIGIT antibody. Suitable antagonistic anti-TIGIT antibodies include monospecific antibodies such as AGEN1327, AB308 (WO2021247591), BMS 986207, COM902, domvanalimab, belrestotug, etigilimab, IBI-929, JS006, dargistotug, ociperlimab, SEA-TGT, tiragolumab, vibostolimab; as well as bi-specific antibodies such as AGEN1777 and rilvegostomig. In certain embodiments, an immune checkpoint inhibitor is an antagonistic anti-TIGIT antibody disclosed in WO2017152088 or WO2021247591. In certain embodiments, an immune checkpoint inhibitor is domvanalimab or AB308.

In some embodiments, an immune checkpoint inhibitor is a LAG-3 antagonist. In further embodiments, the LAG-3 antagonist can be an antagonistic LAG-3 antibody. Suitable antagonistic LAG-3 antibodies include, for example, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody. Suitable antagonist B7-H3 antibodies include, for example, enoblituzumab (MGA271; WO11/109400), omburtumab, DS-7300a, ABBV-155, and SHR-A1811.

In some embodiments, an immune checkpoint inhibitor is a TIM-3 antagonist. In further embodiments, the TIM-3 antagonist can be an antagonistic TIM-3 antibody. Suitable antagonistic TIM-3 antibodies include, for example, sabatolimab, BMS-986258, and RG7769/RO7121661.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and utomilumab (PF-05082566; WO12/32433). In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, the 2-naphthalene sulfonate salt form of Compound (I) is administered in combination with a chemotherapeutic agent and/or an immune checkpoint inhibitor. In some embodiments, the chemotherapeutic agent comprises one or more of a platinum-based, taxoid-based, anthracycline-based chemotherapeutic agent, low-dose cytarabine (LDAC), or gemcitabine. In certain embodiments, the chemotherapeutic agent is selected from cisplatin, carboplatin, oxaliplatin, doxorubicin, docetaxel, paclitaxel, nab-paclitaxel, low-dose cytarabine (LDAC), and gemcitabine. In some embodiments, the chemotherapeutic agent comprises a platinum-based chemotherapeutic agent (e.g., cisplatin, carboplatin, or oxaliplatin). In some embodiments, the chemotherapeutic agent comprises a taxoid-based chemotherapeutic agent (e.g., docetaxel, nab-paclitaxel, or paclitaxel). In some embodiments, the immune checkpoint inhibitor inhibits one or more of PD-1, PD-L1, CTLA-4, LAG-3, TIM-3, and/or TIGIT. In some embodiments, the immune checkpoint inhibitor inhibits PD-1 or PD-L1 (e.g., balstilimab, budigalimab, camrelizumab, cetrelimab, cosibelimab, dostarlimab, cemiplimab, ezabenlimab, MEDI-0680, nivolumab, pembrolizumab, penpulimab, pidilizumab, pimivalimab, retifanlimab, sasanlimab, serplulimab, spartalizumab, sintilmab, tislelizumab, toripalimab, zimberelimab, avelumab, atezolizumab, durvalumab, BMS-936559, and envafolimab). In some embodiments, the 2-naphthalene sulfonate salt form of Compound (I) (e.g., 2-Napsylate Form I) is administered in combination with a taxoid-based chemotherapeutic agent (e.g., docetaxel, nab-paclitaxel, or paclitaxel) and an immune checkpoint inhibitor that inhibits PD-1 or PD-L1. In some embodiments, the 2-naphthalene sulfonate salt form of Compound (I) (e.g., 2-Napsylate Form I) is administered in combination with a taxoid-based chemotherapeutic agent (e.g., docetaxel, nab-paclitaxel, or paclitaxel) and an immune checkpoint inhibitor that inhibits PD-1 or PD-L1 in a subject identified as having STK11 mutant NSCLC.

Selection of the additional therapeutic agent(s) may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Acute Myeloid Leukemia v6.2023, NCCN Squamous Cell Skin Cancer v.1.2023, NCCN Head and Neck Cancers v.1.2024, NCCN Ovarian Cancer/Fallopian Tube Cancer/Primary Peritoneal Cancer v.2.2023, NCCN Pancreatic Adenocarcinoma v.2.2023, NCCN Bladder Cancer v.3.2023, NCCN Mesothelioma: Peritoneal v.2.2023, NCCN Melanoma: Cutaneous v.3.2023, NCCN Melanoma: Uveal vl.2023, NCCN Non-Small Cell Lung Cancer v.4.2023, NCCN Kidney Cancer v.1.2024.

### EXAMPLES

The following examples are put forth so as to provide those of ordinary skill in the art with a complete disclosure and description of how to make and use the present disclosure, and are not intended to limit the scope of what the inventors regard as their disclosure, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: s or sec=second(s); min=minute(s); h or hr=hour(s); d=day(s); w=week(s); equiv=equivalent(s); mg=milligram(s); g=gram(s); μL=microliter(s); ml or mL=milliliter(s); 1 or L=liter(s); mM=millimolar; M=molar; HPLC=high performance liquid chromatography; NMR=nuclear magnetic resonance; XRPD=x-ray powder diffraction; DSC=differential scanning calorimetry; TGA=thermogravimetric analysis; RH=relative humidity; a_{w}=water activity; EtOAc=ethyl acetate; MeOH=methanol; EtOH=ethanol; H₂O=water; ACN=acetonitrile; DCM=dichloromethane; MTBE=methyl tert-butyl ether; CPME=cyclopentyl methyl ether; MEK=methyl ethyl ketone; IPAc=isopropyl acetate; MIBK=methyl isobutyl ketone; DME=dimethoxyethane; IPA=isopropyl alcohol; CHCl₃=chloroform; 2-MeTHF=2-methyl tetrahydrofuran; THF=tetrahydrofuran; DMSO=dimethyl sulfoxide.

### General Techniques

### XRPD

XRPD diffractograms were collected with a Bruker, D2 Phaser or Panalytical Empyrean X-ray diffractometer. The parameters of the XRPD diffraction are given in Tables 1 and 2. Diffractograms can be read by one of skill in the art, for example, to determine if a chemical sample is the substantially the same, different, or represents a mixture of forms. When a diffractogram is “substantially in accordance” as another diffractogram, a person of skill in the art would regard the samples resulting in the two diffractograms as the same form or same mixture of forms, taking into account minor variations for instrumentation, conditions, and the like.

TABLE 1
Bruker, D2 Phaser Data Collection Parameters

| | |
|---|---|
| Instrument | Bruker, D2 Phaser |
| Radiation | Cukα (λ = 1.5418 Å) |
| Detector | LynxEye |
| Scan Angle | 3-40° (2θ) |
| Scan Step | 0.02° (2θ) |
| Scan Speed | 0.2 s/step |
| Tube voltage/current | 30 kV/10 mA |
| Divergence Slit | 1.0 nm |
| Sample Holder | Zero-background sample pan |

TABLE 2
Panalytical Empyrean Data Collection Parameters

| | |
|---|---|
| Instrument | Panalytical Empyrean |
| Radiation | Cukα (λ = 1.5418 Å) |
| Scan Angle | 2-40° (2θ) |
| Scan Step | 0.01° |
| Tube voltage/current | 45 kV/40 mA |
| Scan Speed | 1.31°/minute |

TGA analysis was performed using a thermogravimetric analyzer instrument. About 1-5 mg of sample was loaded onto a pre-tared aluminum pan and heated with the parameters in Table 3. The data was analyzed using TRIOS.

TABLE 3
TGA Data Collection Parameters

| | |
|---|---|
| Instrument | TA, Discovery TGA 55 |
| Sample Pan | Aluminum, open |
| Temperature Range | RT-300° C. |
| Heating Rate | 10° C./min |
| Purge Gas | N₂ |
| Flow Rate | Balance chamber: 40 mL/min |
| | Sample chamber: 25 mL/min |

### DSC

DSC analysis was performed with a thermogravimetric analyzer instrument. About 1-3 mg of sample was placed onto an aluminum pan with pin-holes and heated with the parameters in Table 4. The data was analyzed using TRIOS.

TABLE 4
DSC Data Collection Parameters

| | |
|---|---|
| Instrument | TA, Discovery DSC 250 |
| Sample Pan | Aluminum, pin-holed |
| Temperature Range | 25-300° C. |
| Purge Gas | N₂ |
| Flow Rate | 50 mL/min |

### HPLC

HPLC analysis was performed with an Agilent HPLC 1260 series instrument. HPLC method for solubility and stability testing is summarized in Table 5 below.

TABLE 5
HPLC Method

| Instrument | Agilent 1260 Series | | |
|---|---|---|---|
| Column | Poroshell 120 EC-C18, 2.7 μm, 4.6 × 100 mm | | |
| Column Temperature | 40° C. | | |
| Mobile Phase | A: 0.1% H₃PO₄ in H₂O | | |
| | B: ACN | | |
| Flow Rate | 1.5 mL/min | | |
| Injection Volume | 5 μL | | |
| Detector | DAD | | |
| Wavelength | 254 nm | | |
| Run Time | 10.1 min | | |
| Post Time | 3.0 min | | |
| Diluent | MeOH | | |
| | Time (min) | % A | % B |
| Gradient | 0.0 | 95 | 5 |
| | 6.0 | 60 | 40 |
| | 8.0 | 5 | 95 |
| | 10.0 | 5 | 95 |
| | 10.1 | 95 | 5 |

### Example 1: Synthesis of Compound (I)

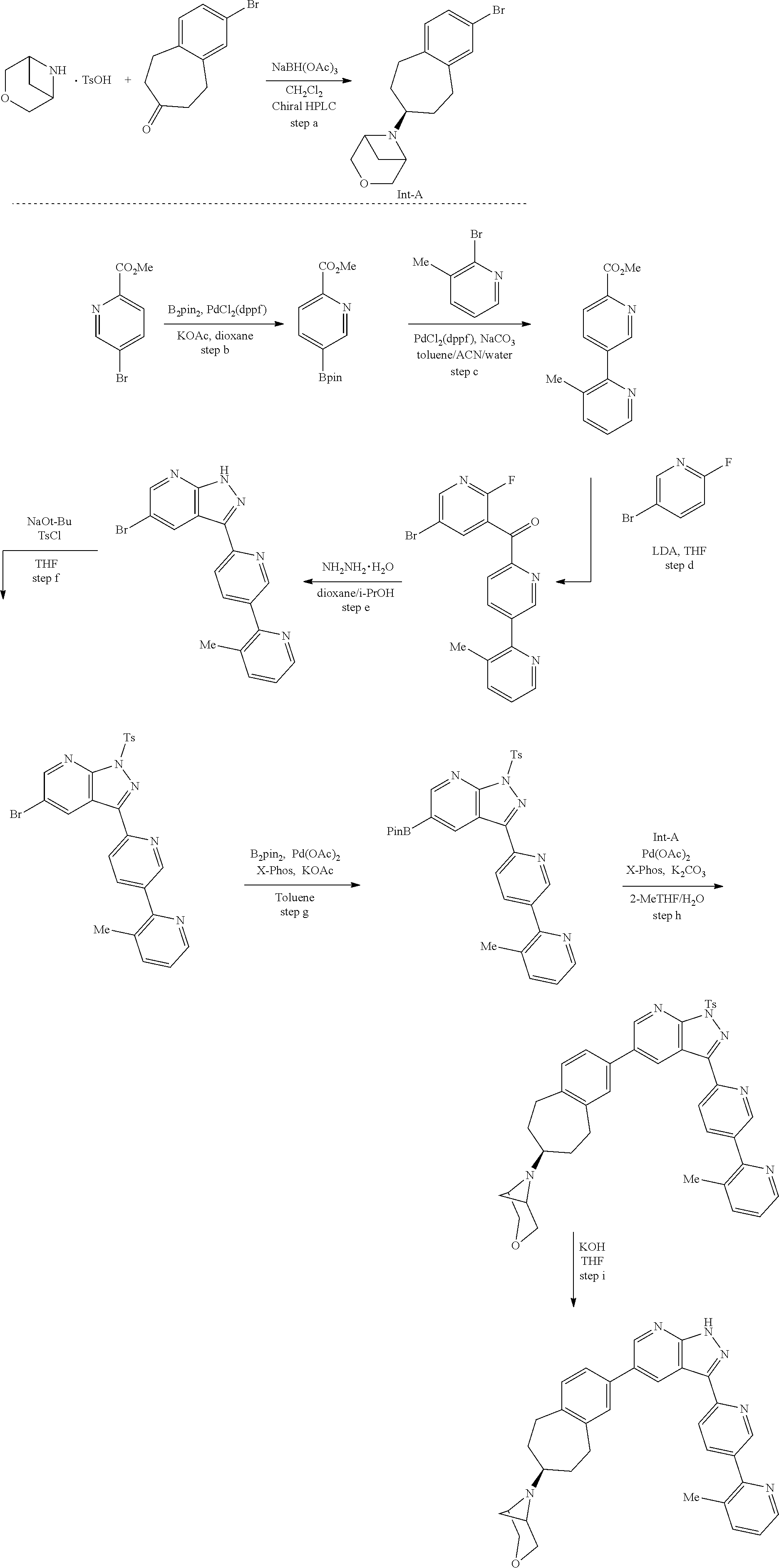

Step a: To a mixture of 3-oxa-6-aza-bicyclo[3.1.1]heptane 4-methylbenzenesulfonic acid in CH₂Cl₂ at room temperature was added 2-bromo-5,6,8,9-tetrahydro-benzocyclohepten-7-one followed by NaBH(OAc)₃. The reaction mixture was then stirred at room temperature for 4 hours. Additional NaBH(OAc)₃ was added at t=4 hours and t=8 hours. After the final addition, the reaction mixture was stirred at room temperature for an additional 4 hours. The reaction mixture was diluted with CH₂Cl₂, water, and 2 M NaOH_{(aq)} (until pH>12), and agitated. The organic phase was washed with 4:1:1 water:brine:2 M NaOH_{(aq)}, dried over Na₂SO₄, and concentrated. The crude material was purified by silica gel chromatography (hexanes:(EtOAc+1% Et₃N) 0 to 100% gradient) to afford the racemic product as a white solid. The racemic mixture was resolved using chiral HPLC (Daicel CHIRALPAK IA; MeOH/DEA 100/0.1 v/v; 1.0 mL/min; UV 230 nm; 5.3 minutes (desired isomer, Int-A), 7.0 minutes (other isomer)).

Step b: To a mixture of methyl 5-bromo-2-pyridinecarboxylate, B₂pin₂, PdCl₂(dppf), and KOAc under nitrogen at room temperature was added degassed dioxane. The reaction mixture was stirred at 100° C. for 1 hour, cooled, diluted with EtOAc, filtered through celite, and concentrated to afford the desired product which was used crude in step b.

Step c: To a mixture of the product from step a, 2-bromo-3-methylpyridine, PdCl₂(dppf), and Na₂CO₃ under nitrogen at room temperature was added degassed toluene, ACN, and water. The reaction mixture was stirred at 70° C. for 2 hours, cooled, diluted with EtOAc, washed with water, washed with brine, dried over Na₂SO₄, and concentrated. The crude material was purified by silica gel chromatography (hexanes:EtOAc 0 to 100% gradient) to afford the desired product as a light brown solid.

Step d: To a solution of 2-fluoro-6-bromopyridine in THF at −78° C. was added LDA (in ethylbenzene/THF/heptane) dropwise. The reaction mixture was stirred at −78° C. for 30 minutes. To the mixture at −78° C. was added a solution of the product from step b in THF. The reaction mixture was stirred at −78° C. for 1 hour, quenched at −78° C. with sat. NH4Cl_{(aq)}, allowed to warm to room temperature, and diluted with EtOAc. The organic phase was dried over Na₂SO₄, concentrated, and triturated with hexane to afford the desired product as a brown solid.

Step e: To a mixture of the product from step c in 1:1 i-PrOH:dioxane at room temperature was added hydrazine monohydrate. The reaction mixture was stirred at room temperature for 1.5 hours. To the mixture at room temperature was added triethylamine and dioxane. The reaction mixture was stirred at room temperature for 3 hours, stirred at 60° C. for 1 hour, diluted with water. The precipitated solids were collected by filtration, washed with water, dried, and recrystallized with EtOAc to afford the desired product as an off-white solid.

Step f: To a mixture of the product from step d and THF at room temperature was added sodium tert-butoxide in one portion. The reaction mixture was stirred at room temperature for 15 minutes and 4-methylbenzenesulfonyl chloride was added in one portion. The reaction mixture was stirred at room temperature for 30 minutes, diluted with water. The precipitated solid were collected by filtration, washed with water, and dried to afford the desired product as brown solid.

Step g: The product of step e, bis(pinacolato)diboron and potassium acetate were mixed in toluene. Palladium acetate and 2-dicyclohexylphosphine-2′,4′,6′-triisopropyl biphenyl were added and the reaction mixture was stirred until completion. Upon reaction completion, the reaction mixture was diluted with dichloromethane and filtered. The supernatant was concentrated and then diluted with n-heptane to form a slurry where the product precipitated. The product was isolated by filtration and the wet cake was dried in vacuo to afford the desired compound.

Step h: The product of step g, Int-A, and potassium carbonate were mixed with 2-methyltetrahydrofuran and water. Palladium acetate and 2-dicyclohexylphosphine-2′,4′,6′-triisopropylbiphenyl were added and the reaction mixture was stirred until completion. Upon reaction completion, the mixture was filtered, and the phases separated. The resulting 2-methyltetrahydrofuran solution was washed with sodium sulfate aqueous solution. The organic solution was then mixed with solid metal scavenger, filtered to remove the scavenger, then optionally filtered through activated carbon cartridge. The solution was then concentrated by distillation and diluted with n-heptane to form a slurry where the product precipitated. The product was isolated by filtration and the wet cake was dried in vacuo to afford the desired compound.

Alternatively, Step g may be performed in 2-methyltetrahydrofuran instead of toluene and telescoped immediately into Step h without isolation of the Step g intermediate. The Step h intermediate can optionally be isolated as a solution in THF instead of as a solid by repeatedly concentrating the washed and filtered 2-methyltetrahydrofuran solution under vacuum with addition of THF. The telescoped procedure and isolation of the Step h intermediate as a solution in THF reduce complexity and improve yield over the two steps.

Step i: An aqueous solution of potassium hydroxide was added to a mixture of the product from step h in tetrahydrofuran. The mixture was stirred until reaction completion and then 2-methyltetrahydrofuran was added followed by a solution of sodium bisulfate. The organic phase was separated, washed with sodium sulfate solution. The organic solution was then mixed with solid metal scavenger, then optionally filtered through activated carbon cartridge, and finally concentrated to give the title compound as an amorphous solid. ¹H NMR (400 MHz, Chloroform-d) δ 11.66 (s, 1H), 9.17 (d, J=2.1 Hz, 1H), 8.94 (dd, J=2.3, 0.9 Hz, 1H), 8.85 (d, J=2.2 Hz, 1H), 8.58 (ddd, J=4.8, 1.7, 0.7 Hz, 1H), 8.31 (dd, J=8.2, 0.9 Hz, 1H), 8.02 (dd, J=8.2, 2.3 Hz, 1H), 7.63 (ddd, J=7.7, 1.7, 0.8 Hz, 1H), 7.44 (dq, J=3.8, 2.0 Hz, 2H), 7.25 (s, 1H), 7.23-7.20 (m, 1H), 4.30 (d, J=10.7 Hz, 2H), 3.73 (d, J=10.7 Hz, 2H), 3.66 (d, J=6.1 Hz, 1H), 3.26 (t, J=9.6 Hz, 1H), 3.07-2.79 (m, 3H), 2.57 (q, J=6.8 Hz, 1H), 2.46 (s, 3H), 1.94 (s, 2H), 1.84 (d, J=8.3 Hz, 1H), 1.41-1.20 (m, 3H), 0.89-0.72 (m, 1H). ESI MS [M+H]⁺ for C₃₃H₃₃N₆O, calcd. 529.3, found 529.3.

### Example 2: Stability of Freebase Compound (I) in Amorphous Form

The stability of the freebase amorphous form of Compound (I), for example, as prepared in Example 1, was assessed by High Performance Liquid Chromatography (HPLC). A reduction in HPLC purity indicates that the sample has undergone chemical degradation. Freebase Compound (I) in amorphous form was added to 5 separate vials. Each vial was sealed and stored for a period of 5 days at 20° C., 40° C., 60° C., 80° C., and 100° C., respectively. Purity was assessed by HPLC initially, and after 1, 3, and 5 days of storage. It was observed that freebase Compound (I) in amorphous form underwent decomposition when stored under the conditions tested. The results from the stability study are summarized in Table 6.

TABLE 6
HPLC Purity of Amorphous Compound (I) (freebase)

| | Day 0 | Day 1 | Day 3 | Day 5 |
|---|---|---|---|---|
| Temp. | (% purity) | (% purity) | (% purity) | (% purity) |
| r.t. | 96.45 | 96.23% | 96.01% | 96.03% |
| 40° C. | 96.45 | 96.12% | 95.87% | 95.00% |
| 60° C. | 96.45 | 95.92% | 93.06% | 92.39% |
| 80° C. | 96.45 | 95.01% | 91.87% | 89.86% |
| 100° C. | 96.45 | 90.13% | 74.66% | 68.00% |

### Example 3: Stability of Amorphous Salt Forms of Compound (I)

The stability of various amorphous salt forms of Compound (I) was also assessed. The salt forms, in general, were prepared by suspending Compound (I) in a suitable solvent at ambient temperature and adding the corresponding acid to the mixture. The mixtures were allowed to stir until a solution was formed, filtered to remove any trace solids, and the resulting filtrate lyophilized to access the salts as solids. Characterization by XRPD established that all salt forms were amorphous. The conditions are summarized in Table 7 below.

TABLE 7
Conditions for Preparation of Amorphous
Acid Addition Salt Forms

| Acid | Equivalents of acid | Solvent |
|---|---|---|
| Citric acid | 1 | water |
| Hydrochloric acid (HCl) | 1 | 4:1 water:acetonitrile |
| Benzoic acid | 1 | 20:7 water:acetonitrile |
| Benzene sulfonic acid | 1 | 4:1 water:acetonitrile |
| Naphthalene sulfonic acid | 1 | 1:1 water:acetonitrile |

The amorphous salt forms were then stored over a period of 14 days at 40° C., and 75% relative humidity (RH). HPLC purity of each sample was assessed initially, and after 14 days of storage to determine the stability of the amorphous salt forms over time. The results are summarized in Table 8 below. Each of the amorphous salt forms showed chemical degradation under the conditions tested.

TABLE 8
HPLC Purity of Amorphous Salt Forms of Compound (I)

| | HPLC Purity Loss on |
|---|---|
| Salt Form Compound (I) (Amorphous) | Day 14 (%) |
| Citrate | 14.2 |
| Chloride | 1.5 |
| Chloride | 6.6* |
| Benzoate | 3.0* |
| Benzene Sulfonic | 3.4 |
| Naphthalene Sulfonate | 2.21 |

*Samples stored at RT for 10 days under ambient humidity (~75% RH); the purity for these samples was calculated as purity on day 3 minus purity on day 10.

### Example 4: Polymorphic Forms of Freebase Compound (I)

### Freebase Form I

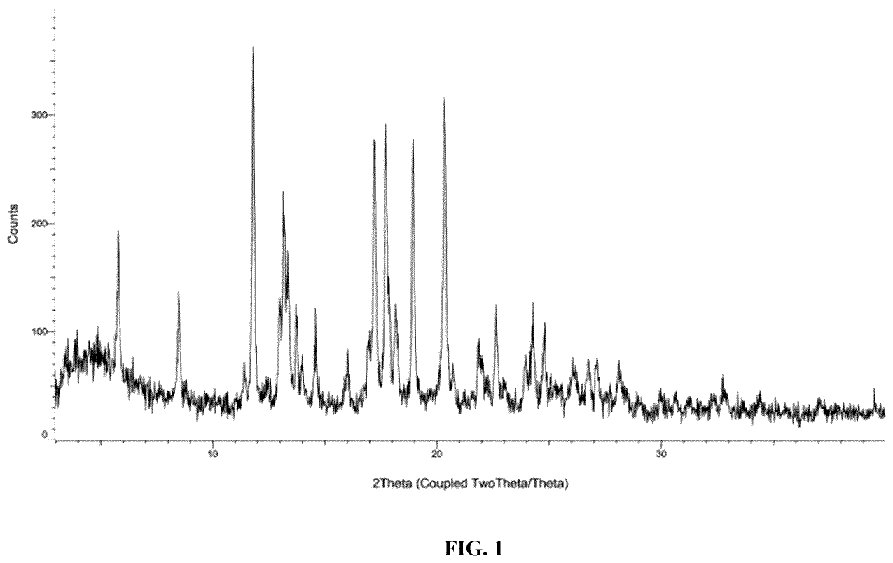

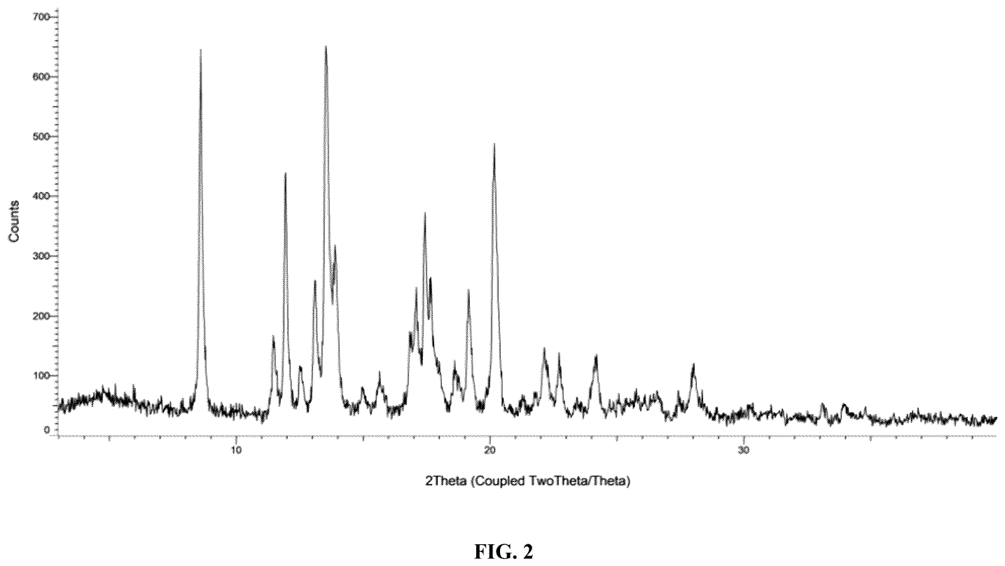

Amorphous compound (I) (30 mg) was added to 500 μL of acetone at RT. The suspension was slurried at RT for 2 h. The solids were collected by filtration and vacuum dried at 40° C. for 15 h. Characterization by XRPD is shown in FIG. 1. The solids contained 7.7% residual acetone (1.1 eq.) as determined by ¹H NMR.

### Freebase Form II

Amorphous compound (I) (300 mg) was added in 16 V of acetone at RT. The suspension was stirred at RT for 2 h. The solids were collected by filtration and vacuum dried at 40° C. for 3 h. The obtained solids were added in 10 V of acetonitrile at 50° C. The suspension was stirred at 50° C. for 3 days. The solids were collected by filtration and vacuum dried at 50° C. for 8 hr. Characterization by XRPD is shown in FIG. 2. The solids contained 0.5% residual acetonitrile and 0.1% acetone as determined by ¹H NMR.

### Freebase Form III

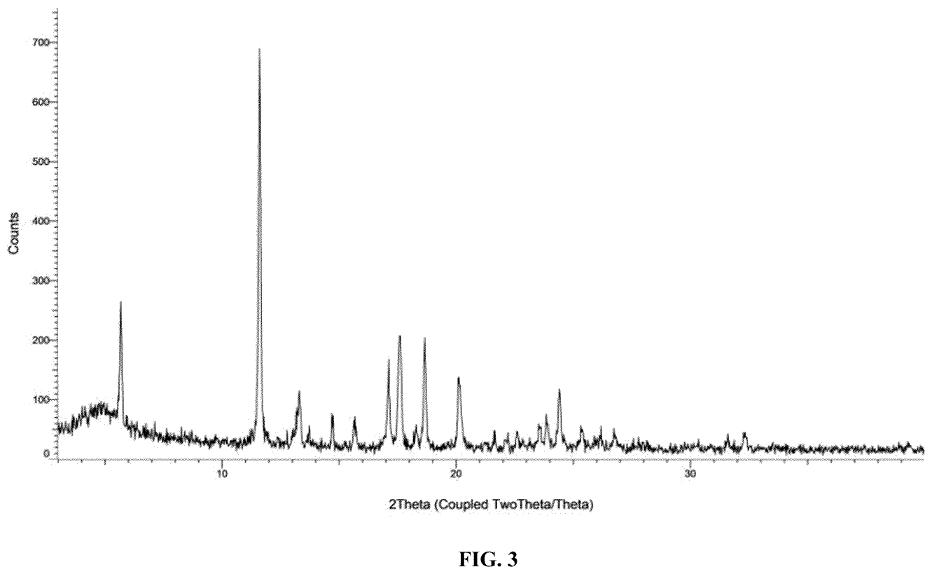

Freebase Form I (20 mg) was added in 1 mL of DMSO at RT. The suspension was slurried at RT for 7 days. The resulting Freebase Form III was collected by filtration and vacuum dried at 40° C. for 3 h. Characterization by XRPD is shown in FIG. 3. The solids contained 13.1% residual DMSO (1 eq.) as determined by ¹H NMR.

A summary of the freebase crystalline forms is provided in Table 9 below.

TABLE 9
Characterization of Crystalline Forms of Compound (I) Freebase

| | | DSC | TGA |
|---|---|---|---|
| Form | Description | Characterization | Characterization |
| Freebase | Acetone solvate | 46° C. (endo) | 25-88° C. |
| Form I | | 113° C. (endo) | (2.5 wt % loss) |
| | | | 88-160° C. |
| | | | (7.9 wt % loss) |
| Freebase | Hydrate | 61° C. (endo) | 32-110° C. |
| Form II | | 130° C. (endo) | (0.9 wt % loss) |
| | | 153° C. (endo) | 111-145° C. |
| | | | (0.6 wt % loss) |
| Freebase | DMSO solvate | 153° C. (endo) | 87-205° C. |
| Form III | | | (11.3 wt % loss) |

### Example 5: Polymorphic Forms of Compound (I) Salts

A variety of acids and a variety of different solvent systems were selected to assess the formation of crystalline material. The acids investigated include HCl, fumaric acid, succinic acid, 2-naphthalene sulfonic acid, camphorsulfonic acid, sulfuric acid, p-toluenesulfonic acid, methanesulfonic acid, phosphoric acid, oxalic acid, L-malic acid, L-tartaric acid, maleic acid, citric acid, benzoic acid, D-malic acid, D-tartaric acid, L-lactic acid, monoethyl succinic acid, benzenesulfonic acid, ethanesulfonic acid, ethane-1,2-disulfonic acid, naphthalene-1,5-disulfonic acid, and isethionic acid. The preparation and characterization of the salt forms identified is described in more detail below.

### 2-Napsylate Form I

2-Naphthalene sulfonic acid (0.95 eq) was dissolved in 180 mL of 2-methyltetrahydrofuran at RT. In a separate reactor, Compound (I) (30 g) was dissolved in 720 mL of n-butanol and heated up to 60° C. To that mixture, 15 mL of 2-naphthalene sulfonic acid solution was added. After an initial batch is made, subsequent reactions are seeded with 2-Napsylate Form I (5% wt., 1.5 g). Remaining amount of 2-naphthalene sulfonic acid solution was slowly added (within 10 h) to the seeded suspension. After addition, the suspension was slurried at 60° C. for 6 h and slowly cooled down to RT and slurried for additional 6 h. The solids were collected by filtration, washed with n-butanol and twice with 2-methyltetrahydrofuran and vacuum dried at RT for 72 h. Characterization by XRPD and DSC is shown in FIG. 4 and FIG. 25, respectively. A summary of the XRPD signal listing is in Table 10.

TABLE 10
XRPD Signal Listing of 2-Napsylate Form I

| Signal (°2θ) | Net Intensity (Counts) |
|---|---|
| 4.2 | 1121 |
| 6.2 | 1378 |
| 7.2 | 63.7 |
| 8.6 | 329 |
| 10.3 | 305 |
| 12.0 | 216 |
| 12.7 | 340 |
| 13.5 | 62.6 |
| 14.1 | 678 |
| 14.6 | 388 |
| 15.3 | 276 |
| 15.7 | 587 |
| 16.1 | 296 |
| 16.5 | 389 |
| 16.8 | 379 |
| 17.5 | 63.0 |
| 18.1 | 111 |
| 18.4 | 1213 |
| 19.1 | 486 |
| 19.4 | 476 |
| 19.9 | 2089 |
| 20.1 | 939 |
| 20.9 | 2130 |
| 21.3 | 412 |
| 21.7 | 135 |
| 22.3 | 157 |
| 22.5 | 264 |
| 22.9 | 601 |
| 23.3 | 268 |
| 23.7 | 200 |
| 24.2 | 147 |
| 24.9 | 234 |
| 25.6 | 185 |
| 26.4 | 303 |
| 27.1 | 172 |
| 27.8 | 62.4 |
| 28.5 | 181 |
| 28.7 | 170 |
| 29.6 | 145 |
| 30.4 | 40.7 |
| 30.8 | 39.9 |
| 31.3 | 33.9 |
| 31.9 | 50.3 |
| 32.9 | 40.8 |
| 33.3 | 26.4 |
| 33.9 | 72.9 |
| 35.9 | 53.1 |
| 36.7 | 64.9 |
| 37.5 | 60.4 |
| 38.1 | 25.4 |
| 38.8 | 43.4 |

### 2-Napsylate Form II

2-Napsylate Form I (40 mg) was added to 400 μL of 1,4-dioxane. The suspension was slurried at RT for 15 h. The solids were collected by filtration and vacuum dried at 40° C. for 3 h. Characterization by XRPD is shown in FIG. 5. The solids contained 10.3% residual 1,4-dioxane (1 eq.) as determined by ¹H NMR. The characterization was consistent with a mono-dioxane solvate.

### 2-Napsylate Form III

2-Napsylate Form I (20 mg) was added to 1 mL of EtOH at RT. The suspension was slurried at RT for 7 days. The solids were collected by filtration and vacuum dried at 40° C. for 3 h. Characterization by XRPD is shown in FIG. 6. The solids contained 6.0% residual EtOH (1 eq.) as determined by ¹H NMR. The characterization was consistent with a mono-EtOH solvate.

### 2-Napsylate Form III*

2-Napsylate Form I (40 mg) was added to 400 μL of IPA at RT. The suspension was slurried at 60° C. for 3 days. The solids were collected by filtration and vacuum dried at 50° C. for 6 h. Characterization by XRPD is shown in FIG. 7. The solids contained 7.5% residual IPA (1 eq.) as determined by ¹H NMR. The characterization was consistent with a mono-IPA solvate. 2-Napsylate Form III* was isomorphic with 2-Napsylate Form III.

### 2-Napsylate Form VI

Amorphous compound (I) (50 g) was added to a reactor and dissolved in 20 V of ethyl acetate. 2-Naphthalene sulfonic acid (1.1 eq) was dissolved in 5 V of ethyl acetate and added dropwise to the reaction solution over 10 minutes. The mixture was stirred at 50° C. for 3 h and 40° C. for 12 h. The resulting mixture was cooled down to 25° C. and the system was concentrated to 10 V under vacuum at 40-45° C. and filtered. The cake was collected and dried under vacuum at 40° C. over 3 h. Characterization by XRPD and DSC is shown in FIG. 8, and FIG. 26, respectively. A summary of the XRPD signal listing is in Table 11.

TABLE 11
XRPD Signal Listing of 2-Napsylate Form VI.

| (°2θ) | Net Intensity (Counts) |
|---|---|
| 4.2 | 3013 |
| 6.3 | 1546 |
| 7.3 | 28.2 |
| 8.6 | 529 |
| 8.8 | 509 |
| 10.5 | 866 |
| 12.2 | 276 |
| 12.8 | 531 |
| 14.2 | 1430 |
| 14.8 | 426 |
| 15.2 | 117 |
| 15.7 | 356 |
| 16.2 | 561 |
| 17.7 | 191 |
| 18.5 | 959 |
| 18.9 | 223 |
| 19.4 | 366 |
| 20.1 | 1133 |
| 21.1 | 1104 |
| 21.4 | 531 |
| 22.4 | 363 |
| 22.9 | 377 |
| 23.3 | 138 |
| 23.9 | 115 |
| 24.3 | 59.0 |
| 24.7 | 132 |
| 25.3 | 407 |
| 25.7 | 62.5 |
| 26.7 | 464 |
| 27.5 | 102 |
| 28.5 | 224 |
| 28.8 | 169 |
| 29.5 | 128 |
| 29.9 | 55.4 |
| 30.2 | 84.2 |
| 32.0 | 78.7 |
| 33.2 | 186 |
| 34.2 | 48.0 |
| 35.2 | 31.1 |
| 35.8 | 56.8 |
| 36.1 | 33.9 |
| 37.0 | 26.8 |
| 37.7 | 54.1 |
| 39.7 | 32.1 |

### Camsylate Form I

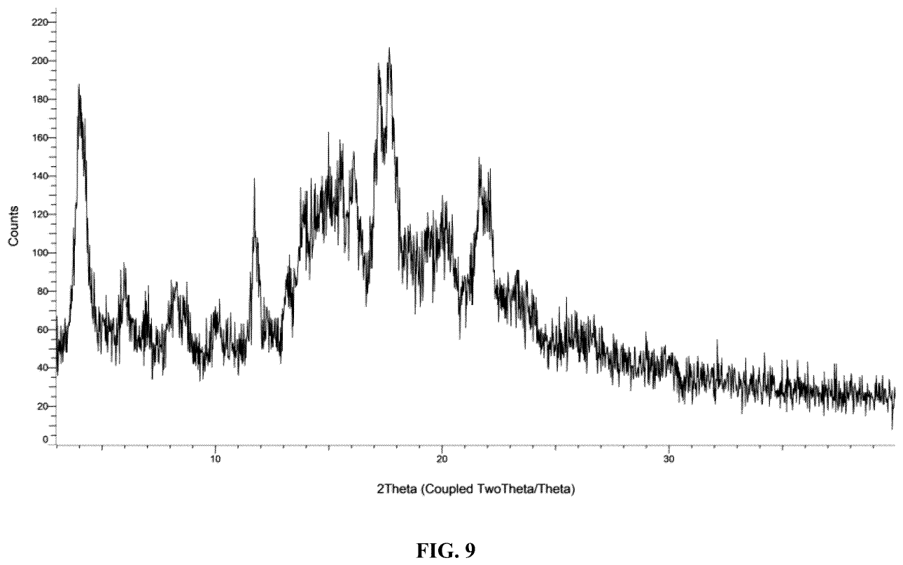

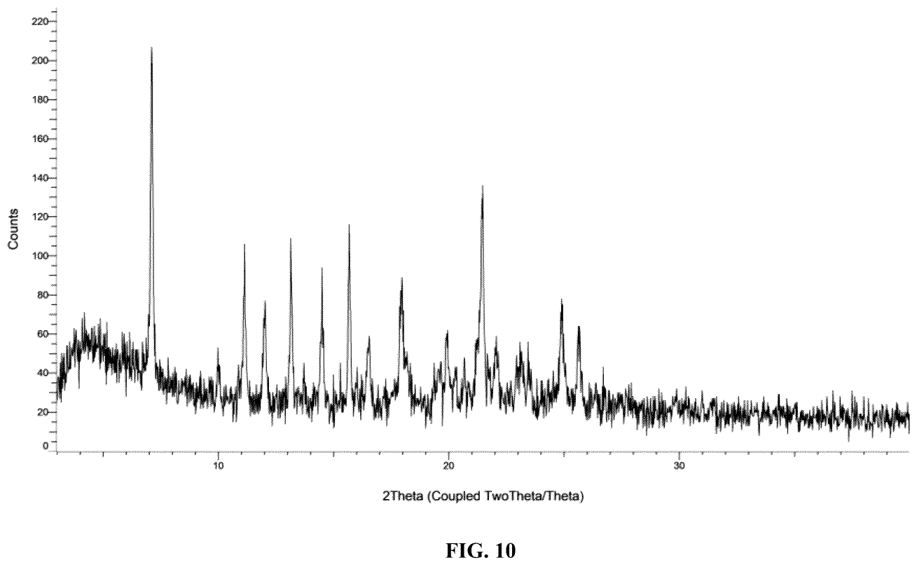

100 mg of Compound (I) was dissolved in 1 mL of acetone. 1.1 equivalents of (1R)-(−)-10-camphorsulfonic acid was added to the solution. The solution was stirred at 40° C. for 1 h and allowed to cool to RT. The suspension was stirred at RT for 1 day. The solids were collected by filtration and vacuum dried at 40° C. for 3 h. Characterization by XRPD is shown in FIG. 9. The solids contained 1.8% residual acetone as determined by ¹H NMR.

### Fumarate Form I

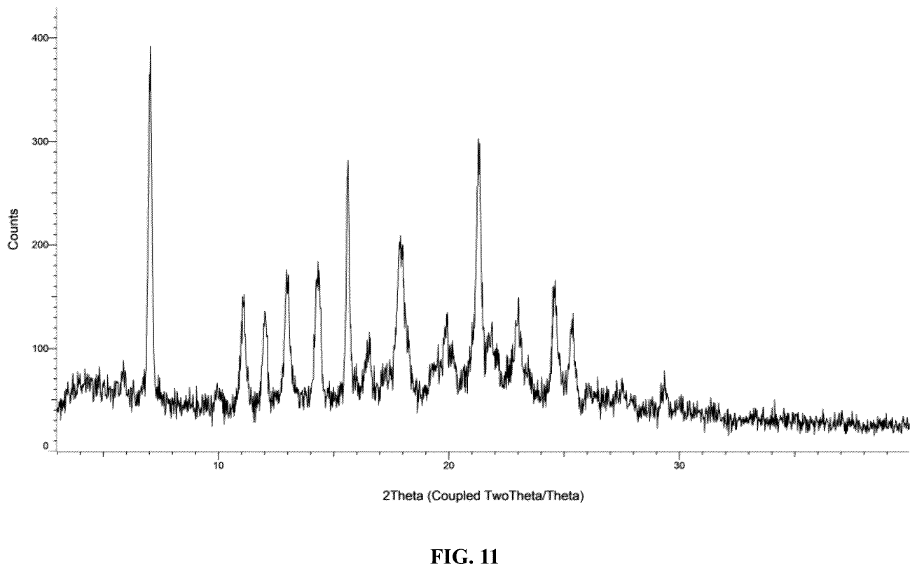

300 mg of Compound (I) were dissolved in 3 mL of EtOH at 50° C. followed by addition of 1.1 equivalents of neat fumaric acid. The solution was stirred at 50° C. for 1 h and cooled to RT. The suspension was stirred at RT for 1 day. The precipitate was collected by filtration, and vacuum dried at 30° C. for 5 h, and then dried under a flow N₂ at RT for another 15 h. Characterization by XRPD is shown in FIG. 10. The solids contained 0.5% residual EtOH as determined by ¹H NMR.

### Succinate Form I

300 mg of Compound (I) was dissolved in 3 mL of acetone at 50° C., and 1.1 equivalents of neat succinic acid was dissolved in the solution. The solution was stirred at 50° C. for 1 h, and cooled to RT followed by being stirred at this temperature for 1 day. The precipitate was collected by filtration and vacuum dried at 30° C. for 5 h, followed by drying under a flow of N₂ for another 15 h. Characterization by XRPD is shown in FIG. 11. The solids contained 1.9% residual acetone as determined by ¹H NMR.

Table 12 summarizes DSC and TGA characterization of the Crystalline Salt Forms (prepared as described above) and using the methods described herein.

TABLE 12
Characterization of Crystalline Salt Forms of Compound I

| Crystalline | Acid/Base | | | TGA |
|---|---|---|---|---|
| Salt Form | Ratio | Description | DSC Characterization | Characterization |
| 2-Napsylate | 1/1 | Hydrate | 247 (exo) | 26-114° C. (2.3 wt % |
| Form I | | | | loss) |
| | | | | 114-188° C. (0.3 wt % |
| | | | | loss) |
| 2-Napsylate | 1/1 | Dioxane solvate | 25-160 (endo) | 25-180° C. (13.3 wt % |
| Form II | | | | loss) |
| 2-Napsylate | 1/1 | EtOH solvate | 183° C. (endo spread) | 29-189° C. (6.0 wt % |
| Form III | | | 194° C. (exo) | loss) |
| 2-Napsylate | 1/1 | IPA solvate | 45° C. (endo) | 78-192° C. (7.5 wt % |
| Form III* | | | 189° C. (endo) | loss) |
| | | | 196° C. (exo) | |
| 2-Napsylate | 1/1 | Hydrate | 243° C. (exo) | 114° C. (2.19 wt % |
| Form VI | | | | loss) |
| | | | | 188° C. (0.28 wt % |
| | | | | loss) |
| Camsylate | 1/1 | Hydrate/solvate | 60° C. (endo) | — |
| Form I | | | | |
| Fumarate | 1/1 | Hydrate | 74° C. (endo) | 27-112° C. (4.7 wt % |
| Form I | | | 149° C. (endo) | loss) |
| Succinate | 1/1 | Hydrate | 65° C. (endo) | 26-115° C. (4.9 wt % |
| Form I | | | 144° C. (endo) | loss) |

### Example 6: Stability of Selected Forms of Compound I

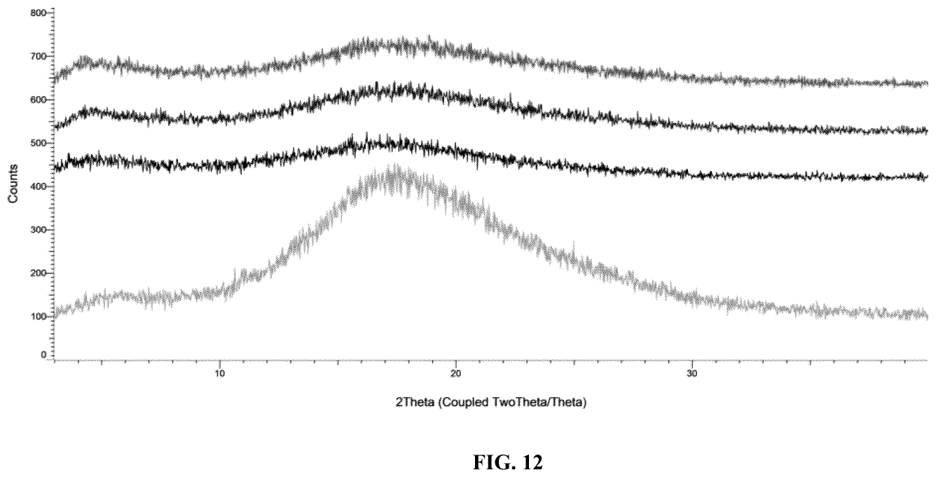

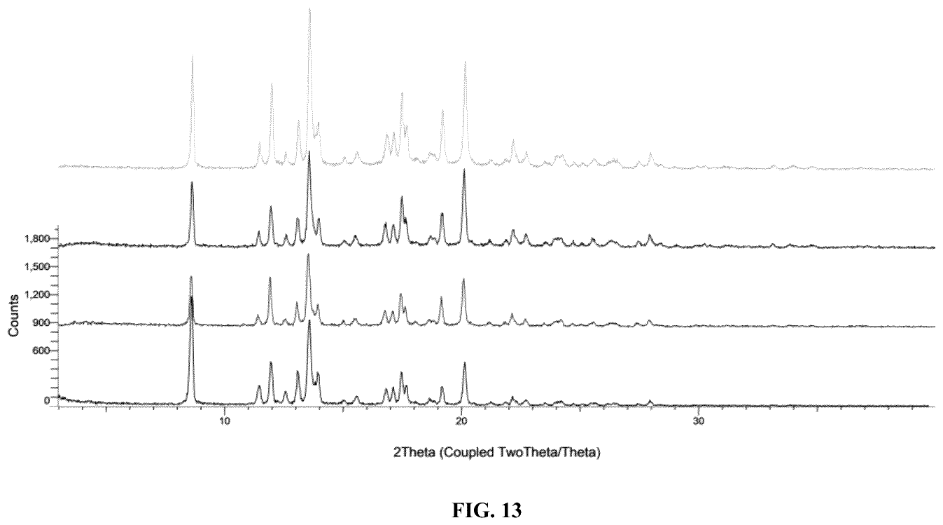

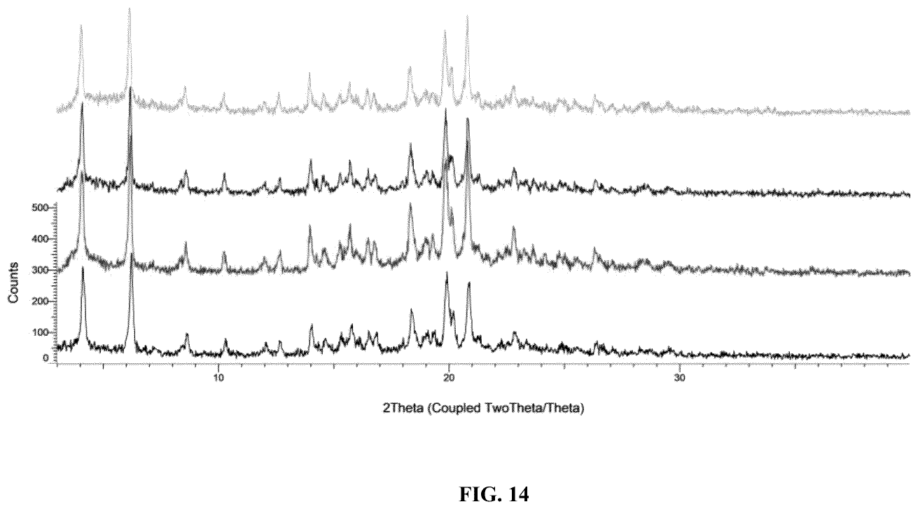

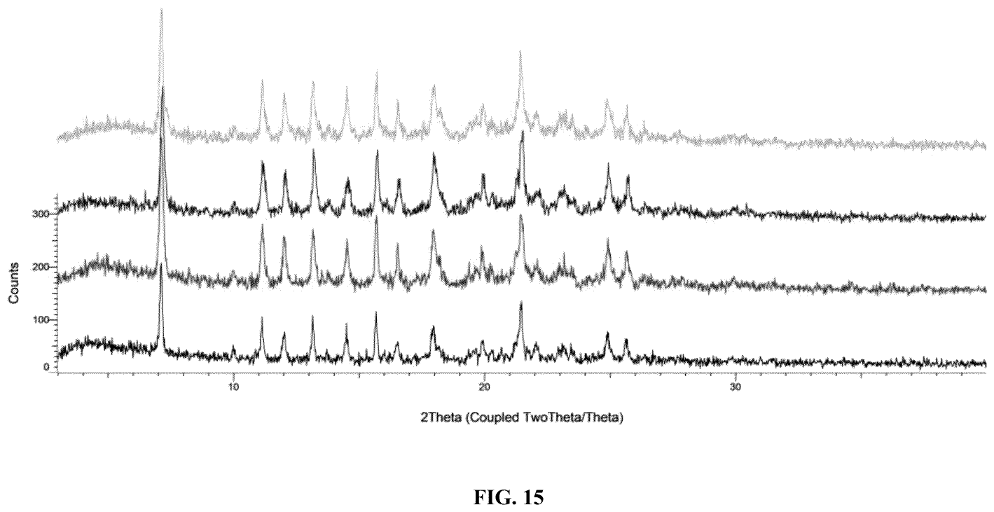

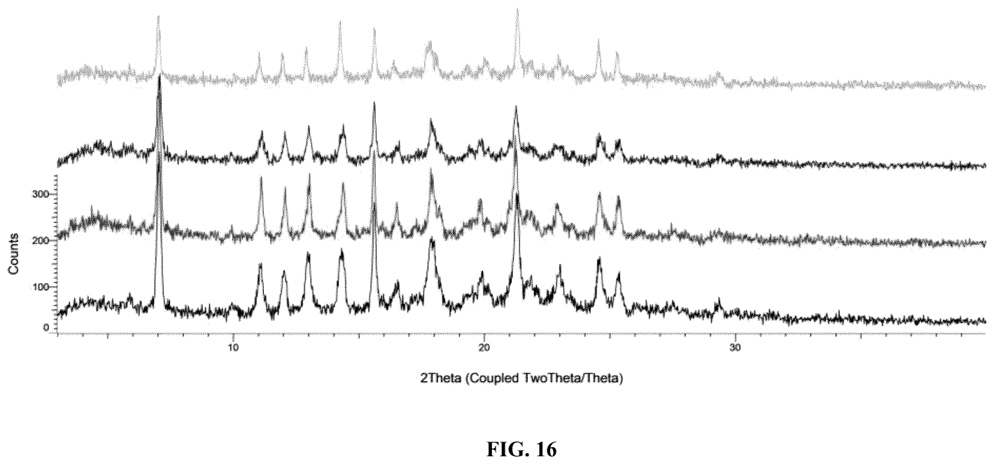

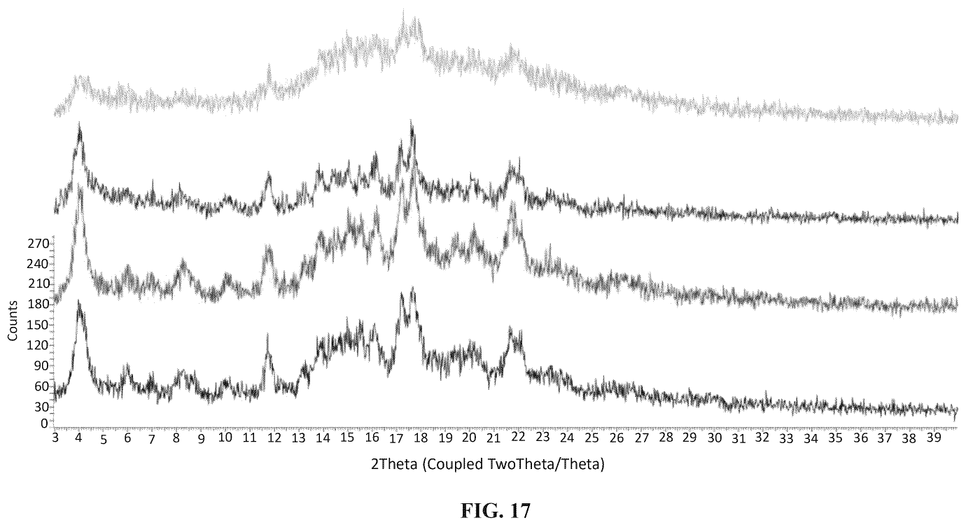

A stability study comparing amorphous freebase Compound I, Freebase Form II, 2-Napsylate Form I, Fumarate Form I, Succinate Form I, and Camsylate Form I was conducted. The experimental results are summarized in Tables 13A-B, and FIGS. 12-17. HPLC measurements were taken after 7 days of storage at the indicated conditions. Freebase Form II and 2-Napsylate Form I showed better chemical and physical stability compared to the other forms tested.

TABLE 13A
HPLC and XRPD Stability of Solid Forms of
Compound I against Amorphous Freebase

| | Amorphous Freebase | | Freebase Form II | | 2-Napsylate Form I | |
|---|---|---|---|---|---|---|
| | HPLC | | HPLC | | HPLC | |
| | Purity | | Purity | | Purity | |
| Condition | (A %) | XRPD | (A %) | XRPD | (A %) | XRPD |
| T₀ | 97.42 | Amorphous | 100.0 | Freebase | 99.82 | 2-Napsylate |
| 40° C. / 75% RH | 96.88 | | 99.36 | Form II | 99.35 | Form I |
| 25° C. | 97.58 | | 99.44 | | 99.82 | |
| 2° C. | 97.68 | | N/A | N/A | N/A | N/A |
| 60° C. | N/A | | 99.22 | Freebase | 99.21 | 2-Napsylate |
| | | | | Form I | | Form I |

TABLE 13B
HPLC and XRPD Stability of Solid Forms of
Compound I against Amorphous Freebase

| | Fumarate Form I | | Succinate Form I | | Camsylate Form I | |
|---|---|---|---|---|---|---|
| | HPLC | | HPLC | | HPLC | |
| | Purity | | Purity | | Purity | |
| Condition | (A %) | XRPD | (A %) | XRPD | (A %) | XRPD |
| T₀ | 97.84 | Fumarate | 98.09 | Succinate | 99.69 | Camsylate |
| | | Form I | | Form I | | Form I |
| 40° C. / 75% RH | 95.29 | | 89.76 | | 99.29 | Decreased |
| | | | | | | crystallinity |
| 25° C. | 97.94 | | 98.22 | | 99.68 | Camsylate |
| 2° C. | 98.01 | | 97.74 | | 99.69 | Form I |
| 60° C. | N/A | N/A | N/A | N/A | N/A | N/A |

### Example 7: 2-Naphthalene Sulfonate Polymorphic Forms

2-Napsylate Form I demonstrated improved chemical and physical stability compared to the other crystalline salt forms tested (Example 6) and was selected for further development. Amorphous material of the Compound (I) 2-naphthalene sulfonate salt was generated by rapid removal of solvent from a solution of the 2-naphthalene sulfonate salt in MeOH using a rotary evaporator at 1.5 g scale. XRPD showed an amorphous pattern. The amorphous material was used for setting up a variety of experiments described in more detail below.

1. Slurry at RT—˜30 mg amorphous material was suspended and stirred in different solvents in 4 mL vials at RT. The solids in the slurry were characterized by XRPD after 7-10 days.
2. Slurry at 50° C./100° C.—˜30 mg amorphous material was suspended and stirred in different solvents in 4 mL vials at 50° C. or 100° C. The solids in the slurry were characterized by XRPD after 4-6 days.
3. Anti-solvent addition—˜30 mg amorphous material was dissolved in different solvents to obtain saturated solutions and anti-solvents were added up to 20 times in volume. The obtained solids were characterized by XRPD.
4. Solid vapor diffusion/humidity induced crystallization—30 mg amorphous material was kept in 4 mL vials which were placed in 20 mL glass vials containing different solvents. The solids were characterized by XRPD after 14 days.
5. Liquid vapor diffusion—˜30 mg amorphous material was dissolved in different solvents to obtain saturated solutions in 4 mL vials which were placed in 20 mL glass vials containing anti-solvents. The obtained solids were characterized by XRPD after 12 days.
6. Slow evaporation at RT and 50° C.—˜30 mg amorphous material was dissolved in different solvents to obtain saturated solutions in 4 mL vials. The vials were covered with paraffin film with 3-5 holes and placed at RT or 50° C. for evaporation. The obtained solids were characterized by XRPD.
7. Slow cooling—˜30 mg amorphous material was dissolved in different solvents to obtain saturated solutions in 4 mL vials at 55° C. using a hot plate. The solutions were slowly cooled down to room temperature. The obtained solids were characterized by XRPD.
8. Polymer-induced crystallization—˜30 mg amorphous material was dissolved in different solvents to create saturated solutions. PVP, PEO, PVA, or HPMC was added to the saturated solutions to induce heteronucleation. The obtained solids were characterized by XRPD after 12 days.
9. Wet grinding—˜30 mg amorphous material was ground with different solvents. The obtained solids were characterized by XRPD.
10. Sonication-induced nucleation—˜30 mg amorphous material was dissolved in low amounts of different solvents in 4 mL vials to obtain clear solutions under heating. The vials were sonicated for one day. The obtained solids were characterized by XRPD.
11. Melt crystallization by additives—˜3-5 mg amorphous material was mixed with additives that have similar melting points and set at melting points in DSC followed by naturally cooling down. The remaining solids were characterized by XRPD.
12. PEG-based crystallization—˜27 mg amorphous material was ground with 3-5 mg PEG-6000/8000. ˜10 mg mixed sample was set in an open pan, heated to 116° C. via DSC, and then kept at 116° C. for 60 minutes. The obtained solids were characterized by XRPD.

The experiments that resulted in crystalline material are summarized in Table 14 below.

TABLE 14
Summary of Experiments Producing Crystalline
2-Napsylate Salt Forms of Compound I

| | | | 2-Napsylate Crystal |
|---|---|---|---|
| Method | Solvent(s) | Observation | Form |
| Slurry at 50° C. | ACN/DMSO (11:1) | Slurry | Form I (converted to |
| | | | Form IV + extra XRPD |
| | | | signals after 20 days) |
| Anti-Solvent Addition | DMF (solvent) | Slurry | Form IV^{#} |
| | THF (anti-solvent) | | |
| Anti-Solvent Addition | DMF (solvent) | Slurry | Form IV^{#} |
| | IPAc (anti-solvent) | | |
| Slow Evaporation | DMF | Yellow Solid | Form VI + Form IV |
| (50° C.) | | | |
| Slow Cooling | DMAc | Yellow Solid^{#} | Form IV |
| Slow Cooling | MeOH/H₂O (1:2) | Yellow precipitate | Form V (converts to |
| | | | Form VII after |
| | | | vacuum drying at 40° |
| | | | C.) |
| Slow Cooling | MeOH/2-MeTHF | Small amount of | Form X (converts to |
| | (6:5) | yellow solid appears | amorphous after |
| | | on the wall | vacuum drying at 40° |
| | | | C.) |

^{#}This sample was evaporated at 50° C.

The characterization details for the identified crystalline 2-naphthalene sulfonate salt forms are summarized in Table 15 below.

TABLE 15
Characterization of Crystalline 2-Naphthalene
Sulfonate Solid Forms of Compound I

| 2-Napsylate | | | | | |
|---|---|---|---|---|---|
| Crystalline | | Wt. Loss in | | Endo. In DSC | |
| Form | Source | TGA (%, ° C.) | | (peak, ° C.) | Description |
| Form I | Example 5 | 2.3 | (26) | 247 | Hydrate |
| | | 0.3 | (114) | | |
| Form II | | 13.32 | (180) | 56.7 | 1,4-Dioxane solvate |
| Form III | | 6.02 | (189) | 183.5 194.1 (exo) | Isomorphic solvate |
| | | | | | (EtOH solvate) |
| Form III* | | 0.22 | (78) | 44.8, 189.3 | Isomorphic solvate |
| | | 8.12 | (192) | 196.3 (exo) | (IPA solvate) |
| Form VI | | 2.19 | (114) | 243 (exo) | Hydrate |
| | | 0.28 | (188) | | |
| Form IV^{#} | Table 14 | N/A | | N/A | Solvate |
| Form V^{&} | | N/A | | N/A | Solvate |
| Form VII | | 2.60 | (200) | 41.4, 84.5, | Hydrate |
| | | | | 166.3, 218.1 | |
| Form VIII\hat{ } | | 1.96 | (200) | 76.8, 223.8 | Hydrate |

| | | | |
|---|---|---|---|
| | | 187.6 (exo) | |
| Form IX^{@~} | N/A | N/A | Solvate/Hydrate |
| Form X^{@} | N/A | N/A | Solvate/Hydrate |

^{#}Form IV converted to Form I after drying at 40° C. under vacuum overnight
^{&}Form V converted to Form VII after drying at 40° C. under vacuum overnight
\hat{ }Form VIII was formed from an attempt to scale up Form VII
^{~}Form IX was isolated from a water activity experiment (Example 9)
^{@}Form IX and X converted to amorphous material after drying at 40° C. under vacuum overnight

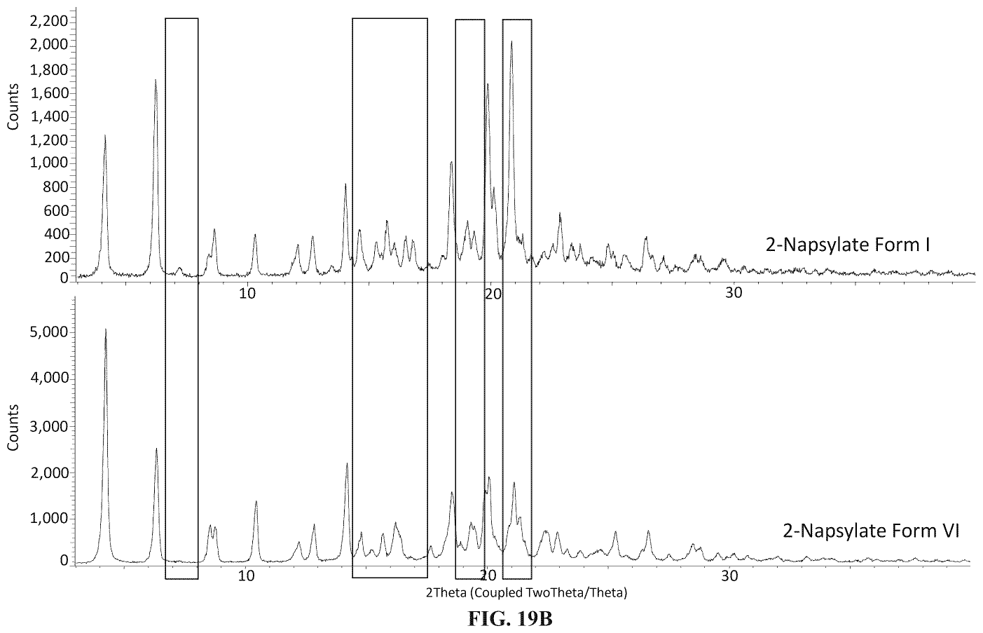

The XRPD characterization of the 2-Napsylate crystalline forms is shown in FIG. 18. A comparison of 2-Napsylate Form I and 2-Napsylate Form VI is shown in FIGS. 19A-19B.

### Example 8: Stability of 2-Napsylate Form I Under Variable Humidity

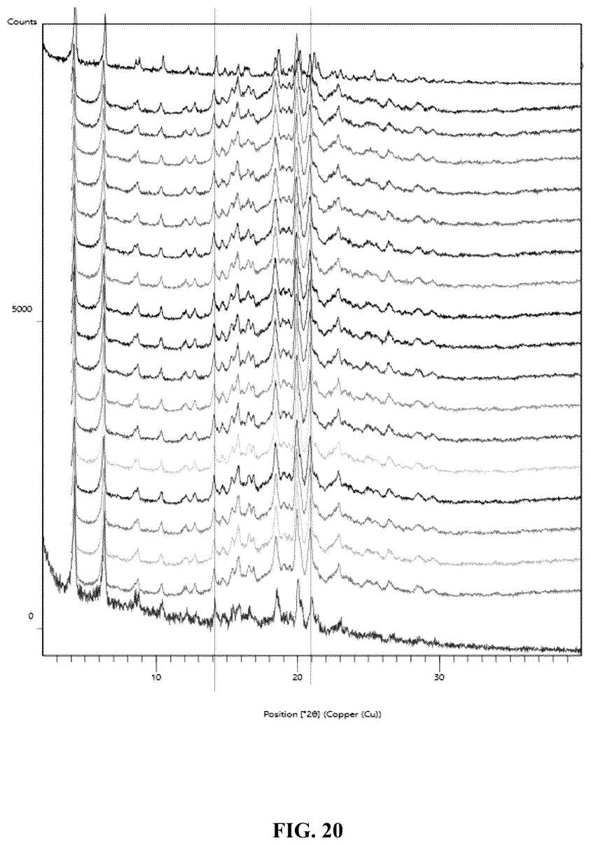

2-Napsylate Form I was used to test form changes as a function of RH from 10% to 90% at 25° C. The XRPD data was collected every 10% RH (FIG. 20) with an equilibration time of 4 hr at each step. No form change was observed at each condition. The results indicate that 2-Napsylate Form I is stable under the variable humidity conditions tested.

### Example 9: Competitive Slurry of 2-Napsylate Form I and 2-Napsylate Form VI

2-Napsylate Form I and 2-Napsylate Form VI were selected for competitive slurry experiments due to their improved crystallinity, stability, and favorable solvent content as compared to the other 2-naphthalene sulfonate crystal forms.

To determine the impact of water activity on 2-Napsylate Form VI and 2-Napsylate Form I, a series of competitive slurry experiments were carried out at different water activities in THF/water and ACN/water solvent systems at RT and 55° C. The results are shown in Tables 16 and 17, respectively. Suspensions of 2-Napsylate Form I at each condition were made and allowed to equilibrate for 2 h followed by addition of ˜5 mg of Form VI with stirring.

TABLE 16
Competitive Slurry a_{w} Experiments with 2-Napsylate
Form I and 2-Napsylate Form VI at RT and 55° C.

| | | |
|---|---|---|
| Solvent | Duration | 2-Napsyalte Crystal Form |

RT

| | | |
|---|---|---|
| THF | 2 d | Form VI (majorly) |
| | 1 w | Form I (majorly) |
| THF/H₂O (a_{w} = 0.2) | 2 d | Form I (majorly) |
| | 1 w | Form I (majorly) |
| THF/H₂O (a_{w} = 0.4) | 2 d | Gel Formation |
| | 1 w | |
| THF/H₂O (a_{w} = 0.6) | 2 d | Gel Formation |
| | 1 w | |
| THF/H₂O (a_{w} = 0.8) | 2 d | Gel Formation |
| | 1 w | |
| H₂O | 2 d | Form I |
| | 1 w | Form IX |

55° C.

| | | |
|---|---|---|
| THF | 2 d | Form VI (majorly) |
| | 1 w | Form I (majorly) |
| THF/H₂O (a_{w} = 0.2) | 2 d | Form I (majorly) |
| | 1 w | Form I |
| THF/H₂O (a_{w} = 0.4) | 2 d | Gel Formation |
| | 1 w | |
| THF/H₂O (a_{w} = 0.6) | 2 d | Gel Formation |
| | 1 w | |
| THF/H₂O (a_{w} = 0.8) | 2 d | Gel Formation |
| | 1 w | |
| H₂O | 2 d | Gel Formation |
| | 1 w | |

TABLE 17
Competitive Slurry a_{w} Experiments with 2-Napsylate
Form I and 2-Napsylate Form VI at RT in ACN

| Solvent | Duration | 2-Napsylate Crystal Form |
|---|---|---|
| ACN | 1 d | Form I |
| ACN/H₂O (a_{w} = 0.2) | 1 d | Form I |
| ACN/H₂O (a_{w} = 0.4) | 1 d | Form I |
| ACN/H₂O (a_{w} = 0.6) | 1 d | Form I |
| ACN/H₂O (a_{w} = 0.8) | 1 d | Gel Formation |
| H₂O | 1 d | Form I |

In the experiments with THF and at water activity (a_{w}) 0.2, 2-Napsylate Form I was obtained after one week at RT as well as 55° C. For the experiment using water at RT, 2-Napsylate Form I was discovered after 2 days, however, it converted to 2-Napsylate Form IX after one week. When the water activity is above 0.2, gel formation was observed except at a_{w} 1 at RT.

Additional water activity experiments were also conducted in ACN/water system. 2-Napsylate Form I was observed in all cases after stirring for 1 day, except at a_{w} 0.8 wherein gel formation was observed.

### Example 10: Hygroscopicity of 2-Napsylate Form I and Form VI

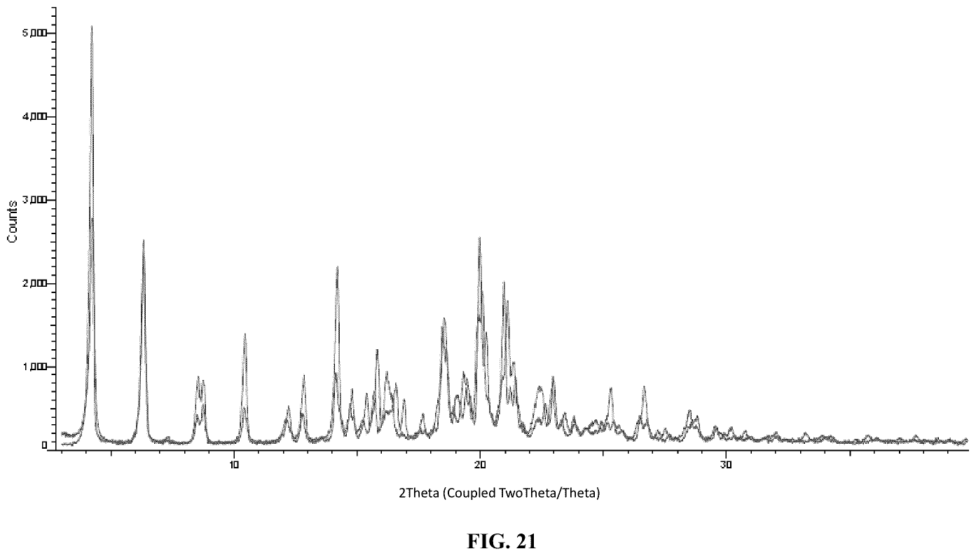

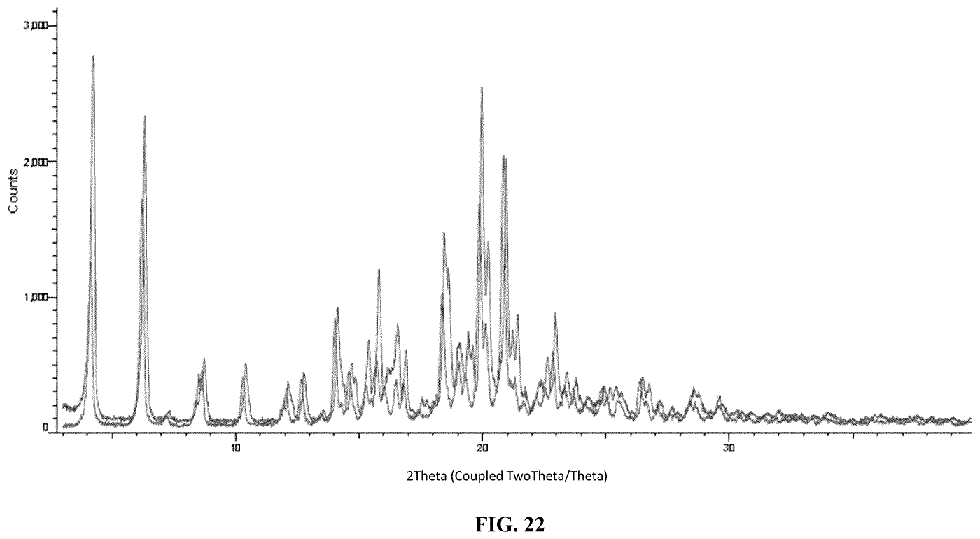

Hygroscopicity of 2-Napsylate Forms I and VI were assessed using DVS. Samples of 2-Napyslate Form I and Form VI (7-20 mg) were placed separately into a tared sample chamber and automatically weighed. The samples were analyzed using the parameters summarized in Table 18 below. XRPD characterization of the samples before and after DVS testing are shown in FIGS. 21-22. XRPD characterization showed no form change of 2-Napsylate Form I after DVS testing.

TABLE 18
Parameters of DVS Analysis

| Instrument | SMS, DVS Intrinsic | |
|---|---|---|
| dm/dt | 0.002%/min | |
| Cycle | Two Cycles | |
| Minimum dm/dt stability duration | 30 | min |
| Maximum dm/dt equilibrium time | 120 | min |
| Save data rate | 5 | s |
| Gas and total flow rate | N₂, 200 sccm | |
| Post experiment total flow | 200 | sccm |

| | |
|---|---|
| RH step size | 10% RH |
| Method | Cycle 1 adsorption: |
| | lab humidity to 90% RH |
| | Cycle 1 desorption: 80, 70, 60, |
| | 50, 40, 30, 20, 10, 0 |
| | Cycle 2 adsorption: 10, 20, 30, |
| | 40, 50, 60, 70, 80, 90 |
| | Cycle 2 desorption: 80, 70, 60, |
| | 50, 40, 30, 20, 10, 0 |

### Example 11: 2-Napsylate Form I Stability

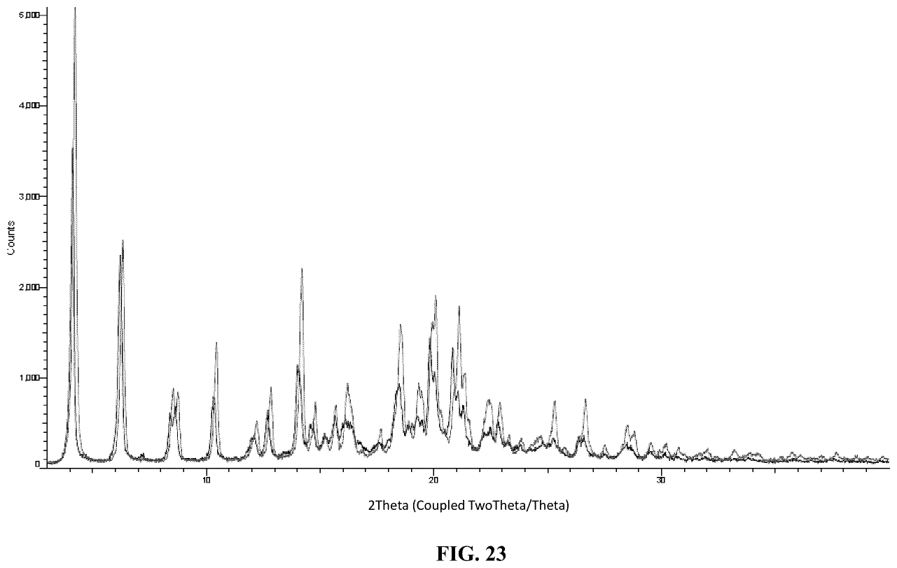

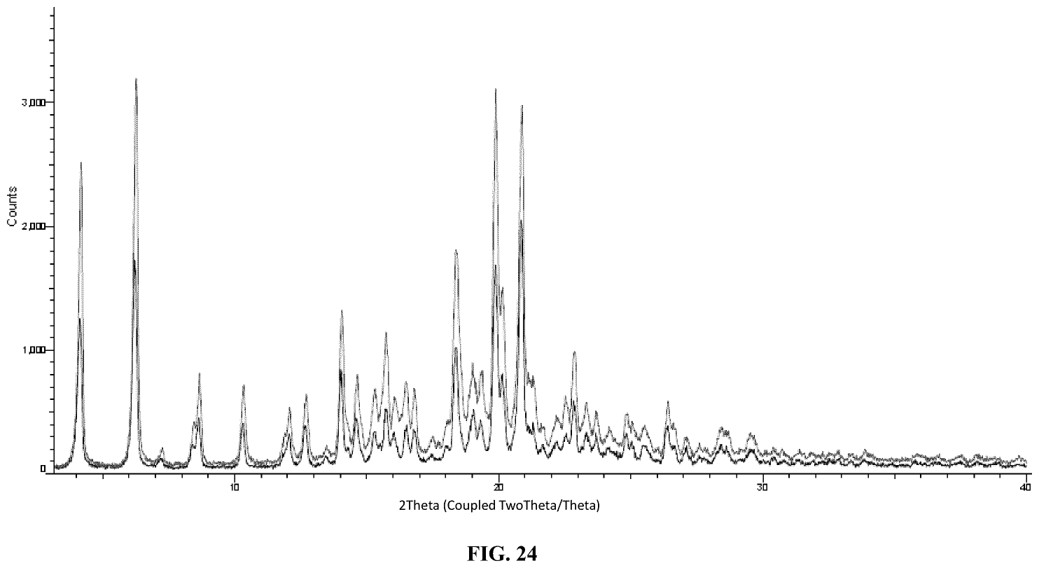

The stability of 2-Napsylate Form I was compared with the stability of 2-Napyslate Form VI when stored at 40° C. and 75% RH for a period of one week. XRPD of the samples before and after storage are shown in FIGS. 23 and 24. The results indicate that 2-Napsylate Form I is stable when stored under the conditions tested.

Samples of 2-Napsylate Form I were further tested for long term and accelerated stability. Samples of the compound are stored at conditions of either 2-8° C. (long term) or 25° C. and 60% RH (accelerated). Stability samples are packaged in double low-density polyethylene (LDPE) bags in a high-density polyethylene (HDPE) outer container.

Samples are tested for appearance, assay, chemical purity, water content, microbial limits, and crystallinity.

As of 3 months into the stability testing, no significant changes to 2-Napyslate Form I were observed when stored at long term (2-8° C.) or accelerated (25° C. and 60% RH) conditions. The stability test protocol is summarized in Table 19.

TABLE 19
Stability Test Protocol for 2-Napsylate Form I

| Storage | Storage Time (Months) | | | | | | | | |
|---|---|---|---|---|---|---|---|---|---|
| Condition | Initial | 1 | 3 | 6 | 9 | 12 | 18 | 24 | 36 |
| Long Term | X, Y, Z | X | X | X, Z^{a} | X | X, Y, Z | X, Z^{a} | X, Y, Z | X, Y, Z |
| (2-8° C.) | | | | | | | | | |
| Accelerated | X | X | X, Y | X, Z | N/A | N/A | N/A | N/A | N/A |
| (25° C. and | | | | | | | | | |
| 60% RH) | | | | | | | | | |

X, Y, and Z indicates tests to be performed: X = appearance, assay, purity, and impurities by HPLC, water content; Y = microbial limits; Z = crystal form;
^{b}optional test

A summary of the stability results of 2-Napsylate Form I when stored at 2-8° C. and 25° C./60% RH are summarized in Tables 20 and 21, respectively.

TABLE 20
HPLC Stability Data for 2-Napsylate Form I at 2-8° C.

| | Time (Months) | | | |
|---|---|---|---|---|
| Test | Initial | 1 | 3 | 6 |
| Assay ‘anhydrous and | 95.8 | 96.9 | 96.1 | 97.7 |
| solvent-free | | | | |
| basis’ (% w/w) | | | | |
| Total impurity (% a/a) | 1.5 | 1.7 | 1.7 | 1.4 |
| Water content (% w/w) | 0.87 | 1.2 | 1.8 | 2.1 |
| Crystallinity | Crystalline | Crystalline | Crystalline | Crystalline |

TABLE 21
HPLC Stability Data form 2-Napsylate
Form I at 25° C. and 60% RH

Time (Months)

| Test | Initial | 1 | 3 | 6 |
|---|---|---|---|---|
| Assay ‘anhydrous and | 95.8 | 96.8 | 95.2 | 96.2 |
| solvent-free basis’ (% w/w) | | | | |
| Total impurity | 1.5 | 1.8 | 1.8 | 1.6 |
| (% a/a) | | | | |
| Water content (% w/w) | 0.87 | 1.2 | 1.6 | 2.1 |

1. A 2-naphthalene sulfonate salt form of Compound (I): 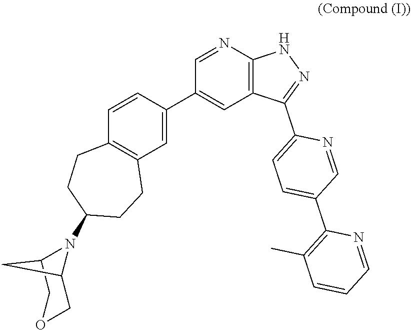 [SMILES: Cc1cccnc1-c1ccc(-c2n[nH]c3ncc(-c4ccc5c(c4)CC[C@@H](N4C6COCC4C6)CC5)cc23)nc1]
or a solvate thereof.

2. The 2-naphthalene sulfonate salt form of claim 1, or solvate thereof, wherein the salt form is a crystalline salt form.

3. The 2-naphthalene sulfonate salt form of claim 1, wherein the salt form is a hydrate.

4. The 2-naphthalene sulfonate salt form of claim 2, wherein the crystalline salt form is characterized by XRPD signals at 4.2, 6.2, 18.4, 19.9, and 20.9 degrees 2θ (±0.2 degrees 2θ), as measured by XRPD using CuKα radiation.

5. The 2-naphthalene sulfonate salt form of claim 4, further characterized by one or more XRPD signals selected from the group consisting of 19.1, 19.4, 21.7, or 22.5 degrees 2θ (±0.2 degrees 2θ), as measured by XRPD using CuKα radiation.

6.-8. (canceled)

9. The 2-naphthalene sulfonate salt form of claim 4, wherein the crystalline salt form is characterized by an XRPD pattern substantially in accordance with FIG. 4; or wherein the crystalline form is characterized by a differential scanning calorimetry (DSC) thermogram substantially in accordance with FIG. 25.

10. The 2-naphthalene sulfonate salt form of claim 4, wherein the crystalline form is characterized by a differential scanning calorimetry (DSC) thermogram having an exotherm signal at about 247° C.

11. (canceled)

12. The 2-naphthalene sulfonate salt form of claim 4, wherein the purity of the salt form decreases by less than 1% as measured by HPLC when stored at:
a. a temperature of about 25° C. for a period of at least 7 days;
b. a temperature of about 60° C. for a period of at least 7 days;
c. a temperature of about 40° C. and a relative humidity of about 75% for a period of at least 7 days,
d. a temperature of about 25° C. and a relative humidity of about 60% for a period of at least 1 month;
e. a temperature between about 2° C. and about 8° C. for a period of at least 1 month;
f. a temperature of about 25° C. and a relative humidity of about 60% for a period of at least 3 months; and/or
g. a temperature between about 2° C. and about 8° C. for a period of at least 3 months.

13-16. (canceled)

17. The 2-naphthalene sulfonate salt form of claim 2, wherein the crystalline form is characterized by XRPD signals at 4.2, 6.3, 14.2, 20.1, and 21.1 degrees 2θ (±0.2 degrees 2θ), as measured by XRPD using CuKα radiation.

18. The 2-naphthalene sulfonate salt form of claim 17, further characterized by one or more XRPD signals selected from the group consisting of 12.2, 21.4, 25.3, and 26.7 degrees 2θ (±0.2 degrees 2θ), as measured by XRPD using CuKα radiation.

19.-21. (canceled)

22. The 2-naphthalene sulfonate salt form of claim 17, wherein the crystalline form is characterized by an XRPD pattern substantially in accordance with FIG. 8: or wherein the crystalline form is characterized by a differential scanning calorimetry (DSC) thermogram substantially in accordance with FIG. 26.

23. The 2-naphthalene sulfonate salt form of claim 17, wherein the crystalline form is characterized by a differential scanning calorimetry (DSC) thermogram having an exotherm signal at about 243° C.

24. (canceled)

25. A pharmaceutical composition comprising a 2-naphthalene sulfonate salt form of claim 1, and one or more pharmaceutically acceptable excipients.

26. A method of treating a disease, disorder, or condition mediated at least in part by AXL, said method comprising administering the 2-naphthalene sulfonate salt form of claim 1 to a subject in need thereof.

27. The method of claim 26, wherein the disease, disorder, or condition is cancer.

28.-30. (canceled)

31. The method of claim 27, wherein said cancer is selected from the group consisting of skin cancer, pancreatic cancer, breast cancer, bladder cancer, liver cancer, lung cancer, colorectal cancer (CRC), leukemia, a brain tumor ovarian cancer, kidney cancer, mesothelioma, and head and neck cancer.

32-35. (canceled)

36. The method of claim 27, further comprising administering one or more additional therapeutic agents to the subject.

37. The method of claim 36, wherein the one or more additional therapeutic agents comprise an immune checkpoint inhibitor, and/or a chemotherapeutic agent.

38.-48. (canceled)

49. A method of treating cancer in a subject in need thereof, said method comprising administering an immune checkpoint inhibitor, a chemotherapeutic agent, and the 2-naphthalene sulfonate salt form of claim 1 to the subject.

50.-69. (canceled)
